(19) United States
(12) Patent Application Publication
(10) Pub. No.: US 2018/0320138 A1
(43) Pub. Date: Nov. 8, 2018

(54) PROCESS OF GENE-EDITING OF CELLS ISOLATED FROM A SUBJECT SUFFERING FROM A METABOLIC DISEASE AFFECTING THE ERYTHROID LINEAGE, CELLS OBTAINED BY SAID PROCESS AND USES THEREOF

(71) Applicants: CENTRO DE INVESTIGACIÓN BIOMÉDICA EN RED (CIBER), Madrid (ES); CENTRO DE INVESTIGACIONES ENERGÉTICAS, MEDIOAMBIENTALES Y TECNOLÓGICAS, Madrid (ES); FUNDACIÓN INSTITUTO DE INVESTIGACIÓ SANITARIA FUNDACIÓN JIMÉNEZ DÍAZ, Madrid (ES); CELLECTIS, Paris (FR)

(72) Inventors: José Carlos SEGOVIA SANZ, Madrid (ES); Oscar QUINTANA BUSTAMANTE, Madrid (ES); Zita Maite GÁRATE MUTILOA, Madrid (ES); Juan Antonio BUEREN RONCERO, Madrid (ES); Brian R. DAVIS, Houston, TX (US); Roman GALETTO, Paris (FR); Agnes GOUBLE, Bois-Collombes (FR); Laurent POIROT, Paris (FR)

(21) Appl. No.: 15/773,960

(22) PCT Filed: Nov. 7, 2016
(86) PCT No.: PCT/EP2016/076893

§ 371 Date: May 4, 2018

(30) Foreign Application Priority Data
Nov. 5, 2015 (EP) 15382545.0

(51) Int. Cl. C12N 5/0789; C12N 5/074

(52) U.S. Cl. CPC C12N 5/0647; C12N 2501/16; C12N 2501/115; C12N 2501/727; C12N 2501/91; C12N 2506/11; C12N 2506/45; C12N 2501/604; C12N 2501/603; C12N 2501/602; C12N 2501/415; C12N 2501/2303; C12N 2501/22; C12N 2501/14; C12N 2501/125; C12N 2501/145; C12N 2501/155; C12N 2501/165; C12N 5/0696

(57) ABSTRACT

The present invention relates to the medical field, in particular to gene editing as a therapeutic approach for the treatment of metabolic diseases affecting the erythroid lineage in a mammalian subject. In invention particular embodiment it refers to the combination of cell reprograming and gene editing for PKD correction as a first example of the potential application of these advanced technologies to metabolic diseases affecting the erythroid lineage. In this sense, PKD patient-specific iPSCs were efficiently generated from PB-MNCs (peripheral blood mononuclear cells) by a SeV non-integrative system and efficiently use to treat pyruvate kinase deficiency. The gene editing strategy for PKLR gene correction was also successfully applied directly to hematopoietic progenitors.

Process of gene-editing of cells isolated from a subject suffering from a metabolic disease affecting the erythroid lineage, cells obtained by said process and uses thereof.

### FIELD OF THE INVENTION

The present invention relates to the medical field, in particular to gene editing as a therapeutic approach for the treatment of metabolic diseases affecting the erythroid lineage in a mammalian subject.

### BACKGROUND OF THE INVENTION

Pyruvate kinase deficiency (PKD; OMIM: 266200) is a rare metabolic erythroid disease caused by mutations in the PKLR gene, which codes the R-type pyruvate kinase (RPK) in erythrocytes and L-type pyruvate kinase (LPK) in hepatocytes. Pyruvate kinase (PK) catalyzes the last step of glycolysis, the main source of ATP in mature erythrocytes (Zanella et al., 2007). PKD is an autosomal-recessive disease and the most common cause of chronic non-spherocytic hemolytic anemia. The disease becomes clinically relevant when RPK activity decreases below 25% of the normal activity in erythrocytes. PKD treatment is based on supportive measures, such as periodic blood transfusions and splenectomy. The only definitive cure for PKD is allogeneic bone marrow transplantation (Suvatte et al., 1998; Tanphaichitr et al., 2000).

However, the low availability of compatible donors and the risks associated with allogeneic bone marrow transplantation limit its clinical application.

### BRIEF DESCRIPTION OF THE INVENTION

In the present invention, we have confronted the problem of providing an alternative treatment for PKD. For this purpose, we have assessed the combination of cell reprogramming and gene editing for PKD correction as a first example of the potential application of these advanced technologies to metabolic diseases affecting the erythroid lineage. In this sense, PKD patient-specific iPSCs have been efficiently generated from PB.MNCs (perypheral blood mononuclear cells) by an SeV non-integrative system. The PKLR gene was edited by PKLR transcription activator-like effector nucleases (TALENs) to introduce a partial codón-optimized cDNA in the second intron by homologous recombination (HR). Surprisingly, we found allelic specificity in the HR, demonstrating the potential to select the allele to be corrected.

### BRIEF DESCRIPTION OF THE FIGURES

FIG. 1: PB-MNC Reprogramming by SeV.

PB-MNCs from healthy donors and PKD patients were reprogrammed by SeV expressing OCT4, SOX2, KLF4, and cMYC mRNAs. Several lines from a healthy donor (PB2iPSC), patient PKD2 (PKD2iPSC), and patient PKD3 (PKD3iPSC) were isolated, expanded, and characterized.
(A) Diagram of the reprogramming protocol.
(B) Representative microphotographs of different iPSC lines derived from PB2 MNC, PKD2 MNC, or PKD3 MNC. Scale bars represent 200 mm.
(C) Sanger sequencing of each patient-specific mutation in the PKLR gene in PB2iPSC, PKD2iPSC, and PKD3iPSC. *Mutations present in patient PKD2. #Mutation present in patient PKD3.

FIG. 2. Gene Editing in the PKLR Locus.
(A) Diagram showing where therapeutic matrix is introduced by HR in the PKLR locus. The strategy to identify the integrated matrix by PCR (horizontal arrows) and Southern blot (vertical arrows) indicating the expected DNA fragment sizes is shown, and the line over the PuroR/thymidine kinase fusion cassette indicates probe location. Small squares at the beginning and end of the partial codon-optimized (cDNA) RPK indicate splicing acceptor and FLAG tag sequences present in the cassette, respectively; light gray squares represent endogenous (mRNA) RPK exons; dark gray squares represent the first LPK exon and 30 UTRs at the beginning and at the end of the PKLR gene, respectively; and black squares represent homology arms.
(B) DNA electrophoresis of gDNA from PuroR-PKD2iPSC clones, amplified by PCR to identify specific matrix integration.
(C) Southern blot of gDNA from edited PKD2iPSC clones, digested by ScaI or SpeI to confirm the precise integration of the matrix in the PKLR locus.

FIG. 3 Allele-Specific Targeting on the PKLR Locus
(A) A single-nucleotide polymorphism (SNP) detected in the second intron of the PKLR gene in PKD2 patient cells, identified by Sanger sequencing. Black arrow points to the polymorphism.
(B) Sequence of PKD2 SNP in the untargeted allele in all the edited PKD2iPSC clones. Letter in red indicates the SNP.
(C) Diagram indicating the position of the SNP with respect to the theoretical cutting site of the PKLR TALEN and the matrix integration in the targeted allele.

FIG. 4. Erythroid Differentiation of PKD2iPSCs

PB2iPSCs, PKD2iPSCs, and edited PKD2iPSCs were differentiated to erythroid cells under specific conditions and analyzed after 31 days in in vitro proliferation and differentiation conditions.
(A) Erythroid differentiation was confirmed by flow cytometry analysis. Cord blood MNCs, PB2iPSC clone c33, PKD2iPC clone c78, and edited PKD2iPSC clone e11 representative analyses are shown.
(B) RPK expression in erythroid cells derived from the different iPSCs was evaluated by qRT-PCR (n=6).
(C) Specific RT-PCR to amplify the chimeric (mRNA) RPK in edited PKD2iPSC. The primers amplified the region around the link between endogenous (mRNA) RPK and the introduced codon-optimized (cDNA) RPK sequence. Arrow indicates the expected band and the corresponding size only preset in the RNA from edited cells (PKD2iPSC ell).
(D) The sequence of the chimeric transcript was aligned with the theoretical expected sequence after the correct splicing between the endogenous exon 2 (blue square) and the exogenous exon 3 (red square).
(E) The presences of RPK protein in erythroid cells derived from PB2iPSCs, PKD2iPSCs, and edited PKD2iPSCs assessed by western blot (upper line); mobility change in PKD2iPSC e11 is due to the FLAG tag added to the chimeric protein. Expression of chimeric protein was detected by anti-FLAG antibody only in erythroid cells derived from edited PKD2iPSCs (bottom line).

FIG. 5. Phenotypic Correction in Edited PKD2iPSCs
(A) ATP levels in erythroid cells derived from healthy iPSCs (PB2iPSCs), PKDiPSCs (patients PKD2 and PKD3), and edited PKDiPSCs (PKD2iPSC e11, PKD3iPSC e88, and PKD3iPSC e31 clones). Data were obtained from three independent experiments from six different iPSC lines derived from two different patients.
(B) In vitro proliferation and differentiation of PB2iPSC clone c33(−), PKD2iPC clone c78(:), and edited PKD2iPSC clone e11
(C). ns, statistically not significant.

FIG. 6. Gene editing of the PKLR locus in hematopoietic progenitors.
(A) Gene editing protocol on hematopoietic progenitors.
(B) Quantification of Hematopoietic Colony Forming Units (CFU) after expansion and without or with puromycin selection. 800 CB-CD34 were seeded per milliliter of HSC-CFU media (Stem Cell Technologies) and two weeks later derived CFUs were counted. All the data were normalized to 5000 seeded CB-CD34. CFUs were counted by observation under a microscope using the 10× and 20× objectives. M: homologous recombination matrix, TM: homologous recombination matrix and PKRL TALEN subunit, CTL (control): CB-CD34 cells nucleofected without adding any nucleic acid material to the media.
(C) Myeloid and erythroid CFUs from CB-CD34 cells transfected, expanded and puromycin selected. Myeloid and erythroid colonies were discriminated based on their morphology and the type of cells forming each colony. Myeloid colonies were white or dark-white formed by granulocytes or monocytes. Erythroid colonies were red or brown formed by erythrocytes.

FIG. 7. Analysis of homologous recombination in CFUs obtained from PKLR gene edited CB-CD34 cells.
(A) Diagram of the Nested PCR designed to analyze gene editing in the PKLR locus.
(B) Nested PCR analysis of CFUs derived from CB-CD34 electroporated with TM and selected with puromycin.
(C) Data from three independent experiments indicating the number of CFU derived from puromycin resistant (Puro^{R}) cells, the number of CFUs positives for homologous recombination analysis and the percentage of gene edited CFUs. All the CFUs were derived from TM transfected and puromycin selected hematopoietic progenitors. No CFU from either CTL or M nucleofected cells were identified. (6d+4d protocol).
(D) Data from two independent experiments indicating the number of CFU derived from puromycin resistant (Puro^{R}) cells obtained after a expansion period of 4 days and puromycin selection of two days (4d+2d protocol).

FIG. 8. Improvement of the delivery of nucleases. Delivery of PKLR TALEN as mRNA.
(A) Diagram of PKLR TALEN mRNA. Both PKLR TALEN subunits were modified by either VEEV 5′UTR (derived from sequence described in Hyde et al, Science 14 Feb. 2014: 783-787), β-Globin 3′UTR or both sequences.
(B) 1×10⁵ CB-CD34 were nucleofected using different amounts of nucleic acids (0.5 m or 2 μg) in a 4D-Nucleofector™ (Lonza) with either PKLR TALEN as plasmid DNA or as in vitro transcribed mRNA carrying different modifications (unmodified mRNA, 5′UTR VEEV mRNA and 3′UTR b-Globin mRNA). Surveyor assay (IDT) to determine the ability of the different nucleases to generate insertions and deletions (indels) in the PKLR locus target site was performed three days after electroporation (left panel) or in CFUs derived from nucleofected hematopoietic progenitors (right panel).
(C) Quantification of indels obtained in the surveyor assays showed in B evaluated by band densitometry and ratio of band intensities between cleaved and uncleaved bands (%).

FIG. 9. Gene edition of the PKLR locus on NSG Engrafted Hematopoietic Stem Cells.
(A) Diagram of gene editing analysis in human Hematopoietic Stem Cells after engrafting in NSG mice. Fresh CB-CD34 cells were nucleofected by the HR matrix plus either PKLR TALEN as plasmid DNA or mRNA. The cells were cultured and puromycin selected. Selected CB-CD34 cells were transplanted intravenously in sub-lethally irradiated immunodeficient NSG mice (NOD.Cg-Prkdc^{scid}II2rg^{tm1Wjl}/SzJ). Four months after transplantation, human engraftment was analyzed by FACS to identify i) human hematopoieitc cells (hCD45⁺) over mouse hematopoietic cells (mCD45⁺) and ii) human hematopoietic progenitors (CD45⁺/CD34⁺). CD45⁺/CD34⁺ cells were then isolated from the mouse bone marrow by cell sorting. Isolated human progenitors were cultured, puromycin selected as indicated in FIG. 7C and CFU assay was performed thereafter. Gene editing in these engrafted human hematopoietic progenitors was analyzed in individual CFUs by Nested PCR as shown in FIG. 7A.
(B) FACS analysis of human hematopoietic engraftment in the bone marrow of NSG after four months post-transplantation. Left panels, human engraftment in NSG mice transplanted with CB-CD34 nucleofected with the matrix and PKLR TALEN as DNA; right panels, human engraftment in NSG mice transplanted with CB-CD34 nucleofected with the matrix and PKLR TALEN as mRNA;
(C) Gene editing analysis by nested PCR in engrafted human hematopoietic progenitors in NSG mice, after enrichment with cell sorting for hCD45⁺CD34⁺cells and another puromycin treatment. CFUs derived from engrafted human CD34 were positive for HR when the gene edition was mediated by electroporation of PKLR TALEN as mRNA.

### DETAILED DESCRIPTION OF THE INVENTION

Herein, we have shown the potential to combine cell reprograming and gene editing as a therapeutic approach for PKD patients. We generated iPSCs from PB-MNCs taken from PKD patients using a non-integrating viral system. These PKDiPSC lines were effectively gene edited via a knock-in strategy at the PKLR locus, facilitated by specific PKLR TALENs. More importantly, we have demonstrated the rescue of the disease phenotype in erythroid cells derived from edited PKDiPSCs by the partial restoration of the step of the glycolysis affected in PKD and the improvement of the total ATP level in the erythroid cells derived from PKDiPSCs. The restoration of the energetic balance in erythroid cells derived from PKD patients opens up the possibility of using gene editing to treat PKD patients.

To reprogram patient cells, we adopted the protocol of using a patient cell source that is easy to obtain, PB-MNCs, and an integration-free reprogramming strategy based on SeV vectors (sendai viral vector platform). PB-MNCs were chosen, as blood collection is common in patient follow-up and is minimally invasive. Additionally, it is possible to recover enough PB-MNCs from a routine blood collection to perform several reprogramming experiments. Finally, previous works showed that PB-MNCs could be reprogrammed, although at a very low efficiency (Staerk et al., 2010). On the other hand, the SeV reprogramming platform has been described as a very effective, non-integrative system for iPSC reprogramming with a wide tropism for the target cells (Ban et al., 2011; Fusaki et al., 2009). Reprogrammed SeVs are cleared after cell reprogramming due to the difference of replication between newly generated iPSCs and viral mRNA (Ban et al., 2011; Fusaki et al., 2009). However, reprogrammed T or B cells might be favored when whole PB-MNCs are chosen, as these are the most abundant nucleated cell type in these samples. Reprogramming Tor B cells has the risk of generating iPSCs with either TCR or immunoglobulin rearrangements, decreasing the immunological repertoire of the hematopoietic cells derived from these rearranged iPSCs. In order to avoid this possibility, we have biased the protocol against reprogramming of either T or B lymphocytes by culturing PB-MNCs with essential cytokines to favor the maintenance and proliferation of hematopoietic progenitors and myeloid cells. This approach was supported here by the demonstration that SeV vectors preferentially transduced hematopoietic progenitors and myeloid cells under these specific conditions and consequently none of the iPSC lines analyzed had immunoglobulin or TCR re-arrangements. We further demonstrated that the generation of iPSCs from PB-MNCs using SeV is feasible and simple and generates integration-free iPSC lines with all the characteristic features of true iPSCs that could be further used for research or clinical purposes.

The next goal for gene therapy is the directed insertion of the therapeutic sequences in the cell genome (Garate et al., 2013; Genovese et al., 2014; Karakikes et al., 2015; Song et al., 2015). A number of different gene-editing strategies have been described, including gene modification of the specific mutation, integration of the therapeutic sequences in a safe harbor site, or knock-in into the same gene locus. We directed a knock-in strategy to insert the partial cDNA of a codon-optimized version of RPK in the second intron of the PKLR gene. If used clinically, this strategy would allow the treatment of up to 95% of the patients, those with mutations from the third exon to the end of the (cDNA) RPK (Beutler and Gelbart, 2000; Fermo et al., 2005; Zanella et al., 2005). Additionally, this approach retained the endogenous regulation of RPK after gene editing, a necessary factor as RPK is tightly regulated throughout the erythroid differentiation. This fine control would be lost if a safe-harbor strategy was chosen.

The PKLR TALEN generated was very specific and very efficient. We did not find any mutation in any of the theoretical off-target sites defined by the off-site search algorithm and analyzed by PCR and gene sequenced. Moreover, we determined that 2.85 out to 100,000 electroporated PKDiPSCs, without considering the toxicity associated to nucleofection, were gene edited when the PKLR TALEN was used, reaching values similar to those previously published by others (Porteus and Carroll, 2005). Interestingly, 40% of the edited PKDiPSC clones presented indels in the untargeted allele or were biallelically targeted, which indicated that the developed TALEN are very efficient, cutting on the on-target sequence with a high frequency.

Surprisingly, we found that the presence of a single SNP 43 bp away from the PKLR TALEN cutting site was an impediment to HR. Taking into account that the TALEN cut has occurred, as we can detect indels in the non-targeted allele, the absence of matrix insertion seems to be directly related to problems related with the perfect annealing of the matrix with the genome sequences. We have to point out that this SNP is located in a very repetitive region, which might form a structural configuration that increases the HR specificity between this region and its homology arm, as has already been mentioned (Renkawitz et al., 2014). Thus, the genome context where the HR has to take place plays an important role and can facilitate or impair HR. In any case, these data demonstrate the important need for gene-editing strategies to generate the homology arms of an HR matrix from the individual DNA that will be edited. This would restrict HR matrices to patients with similar SNPs in the genomic region to be edited. Therefore, any gene-editing therapy using a knock-in or safe-harbor strategy should first screen each patient for the presence of an SNP in the homology arms selected. On the other hand, the presence of a specific SNP could also help to perform allele-specific gene targeting in the cases where the presence of a dominant allele is pathogenic as, for example, in α-thalassemia (De Gobbi et al., 2006).

The gene-editing strategy utilized here to correct PKD was safe, since neither the introduction of genomic alterations nor alteration of the expression of neighboring genes by the insertion and expression of the exogenous sequences occurred. This demonstrates the safety of this knock-in gene-editing strategy without cis activation of any gene, in comparison to previous results where the selection cassette deregulated nearby genes (Zou et al., 2011). Furthermore, we did not observe any off-target effects induced by PKLR TALEN gene editing.

We found several genomic alterations by CGH and exome sequencing analysis. However, the majority of them were already present in PKD PB-MNCs before their reprogramming, especially in the case of the biallelic targeted PKD3iPSC c31, where all of the CNVs were already present in PKD3iPSC c54, confirming previous data associating these DNA variations in iPSC clones with a cellular mosaicism in the original samples (Abyzov et al., 2012). However, there were some mutations present in the iPSC that we were unable to detect in the original sample, which might be due to technical limitations or to the inherent genetic instability associated with the reprogramming process and iPSC culture (Gore et al., 2011; Hussein et al., 2011). Supporting this last possibility, we found CNVs present in PKD2iPSC c78 and not in PKD2iPSC e11 (Table 2). Because PKD2iPSC c78 was maintained in vitro for several more passages, after HR and before CGH analysis, some new changes could have occurred that were not present in the gene-edited-derived clones. Although one CNV involved the TCEA1 gene, indirectly involved in salivary adenoma as a translocation partner of PLAG1 (Asp et al., 2006), none of these genomic alterations identified were implicated in hematopoietic malignancies, cell proliferation, or apoptosis regulation, suggesting their neutrality in the PKD therapy by gene editing.

Constitutive expression of Puro/TK from the ubiquitously active mPGK promoter might hinder therapeutic applications of this approach. Indeed, these highly immunogenic prokaryotic/viral proteins can be presented on the cell surface of the gene-corrected cells by the major histocompatibility complex class I molecules, thus stimulating an immune response against the cells once transplanted into the patients. Here, although the Puro/TK cassette has been maintained in the edited PKDiPSC lines, the cassette is inserted between two loxP sites, which would allow us to excise it before their clinical application. Moreover, for the potential clinical use of our approach, other selection systems could be used, such as a truncated version of the nerve growth factor receptor combined with enrichment by magnetic sorting, or the use of an inducible or an embryonic-specific promoter instead of the PGK constitutive promoter to limit the Puro/TK expression.

Finally, we have clearly demonstrated the effectiveness of editing the PKLR gene in PKDiPSCs to recover the energetic balance in erythroid cells derived from edited PKDiPSCs. ATP and other metabolites involved in glycolysis were restored by expressing a chimeric RPK in a physiological manner. Erythroid cells derived from monoallelic corrected PKDiPSCs produce partial restoration of ATP levels, and erythroid cells derived from biallelic corrected PKD3iPSC e31 fully recovered ATP level (FIG. 5A). Additionally, we could not observe any difference in the erythroid populations obtained in vitro from uncorrected and corrected PKDiPSCs, probably due to the lack of terminal differentiation/enucleation of the protocol used to generate mature enucleated erythrocytes. Furthermore, we were able to generate 20,000 erythroid cells per starting iPSC, providing abundant material for our assays and offering the potential to undertake the therapeutic usage of these cells.

In summary, we combined gene editing and patient-specific iPSCs to correct PKD. Our gene-editing strategy was based on inserting a partial codon-optimized (cDNA) RPK in the PKLR locus mediated by PKLR TALEN without altering the cellular genome or neighbor gene expression. Additionally, we found highly homologous sequence specificity, since a single SNP could avoid HR. The resultant edited PKDiPSC lines could be differentiated to large number of erythroid cells, where the energetic defect of PKD erythrocytes was effectively corrected. This validates the use of iPSCs for disease modeling and demonstrates the potential future use of gene editing to correct PKD and also other metabolic red blood cell diseases in which a continuous source of fully functional erythrocytes is required.

In addition, the inventors have shown that the gene editing strategy successfully used with iPSCs can also be applied directly to human hematopoietic progenitors, which provides the advantage of avoiding the step of reprogramming the iPSCs into hematopoietic progenitors further to the gene editing process. In particular, specific integration of the therapeutic matrix in the PKLR locus was shown to correct the defect in the PKLR gene also in hematopoietic progenitors (Examples 9 and 10). Improved results where obtained when PKLR TALEN subunit was transfected as 5′ and/or 3′ modified mRNA (Examples 11 and 12).

Therefore, a first aspect of the invention, refers to cells which have the ability to differentiate into the erythroid lineage, such as i) hematopoietic stem or progenitor cells or ii) induced pluripotent stem cells obtained from adult cells (Li et al.,2014), preferably derived from peripheral blood mononuclear cells, isolated from a mammalian subject, preferably from a human subject, suffering from a metabolic disease affecting the erythroid lineage, wherein the mutation or mutations in the gene causing said metabolic disease are corrected by gene-editing of the induced pluripotent stem cells obtained from adult cells via a knock-in strategy, where a partial cDNA is inserted in a locus of the target gene to express a chimeric mRNA formed by endogenous first exons and partial cDNA under the endogenous promoter control.

The term “cells” and “cell population” are used interchangeably. The term “cell lineage” as used herein refers to a cell line derived from a progenitor or stem cell, including, but not limited to a hematopoietic stem or progenitor cell.

Hematopoietic cells are typically characterized by being (CD45⁺) and human hematopoietic stem or progenitor cells CD45⁺ and CD34⁺. The term “hematopoietic stem cells” as used herein refers to pluripotent stem cells or lymphoid or myeloid stem cells that, upon exposure to an appropriate cytokine or plurality of cytokines, may either differentiate into a progenitor cell of a lymphoid or myeloid cell lineage or proliferate as a stem cell population without further differentiation having been initiated. Hematopoietic stem or progenitor cells may be obtained for instance from bone marrow, umbilical cord blood, placenta or peripheral blood. It may also be obtained from differentiated cell lines by a cell reprogramming process, such as described in WO2013/116307.

The terms “progenitor” and “progenitor cell” as used herein refer to primitive hematopoietic cells that have differentiated to a developmental stage that, when the cells are further exposed to a cytokine or a group of cytokines, will differentiate further to a hematopoietic cell lineage. “Progenitors” and “progenitor cells” as used herein also include “precursor” cells that are derived from some types of progenitor cells and are the immediate precursor cells of some mature differentiated hematopoietic cells. The terms “progenitor” and “progenitor cell” as used herein include, but are not limited to, granulocyte-macrophage colony-forming cell (GM-CFC), megakaryocyte colony-forming cell (CFC-mega), burst-forming unit erythroid (BFU-E), colony-forming cell-megakaryocyte (CFC-Mega), B cell colony-forming cell (B-CFC) and T cell colony-forming cell (T-CFC). “Precursor cells” include, but are not limited to, colony-forming unit-erythroid (CFU-E), granulocyte colony forming cell (G-CFC), colony-forming cell-basophil (CPC-Bas), colony-forming celleosinophil (CFC-Eo) and macrophage colony-forming cell (M-CFC) cells.

The progenitors and precursor cells according to the first aspect of the invention are those of the erythroid lineage, namely myeloid and erythroid progenitor cells which includes burst-forming unit erythroid (BFU-E) and colony-forming unit-erythroid (CFU-E).

The term “cytokine” as used herein further refers to any natural cytokine or growth factor as isolated from an animal or human tissue, and any fragment or derivative thereof that retains biological activity of the original parent cytokine. The cytokine or growth factor may further be a recombinant cytokine or recombinant growth factor. The term “cytokine” as used herein refers to any cytokine or growth factor that can induce the differentiation of a cell with stem cell properties, such as from an iPSC or a hematopoietic stem cell to a hematopoietic progenitor or precursor cell and/or induce the proliferation thereof. Suitable cytokines for use in the present invention include, but are not limited to, erythropoietin (EPO), granulocyte-macrophage colony stimulating factor (GM-CSF), granulocyte colony stimulating factor (G-CSF), macrophage colony stimulating factor (M-CSF), thrombopoietin (TPO), stem cell factor (SCF), interleukin-1 (IL-1), interleukin-2 (IL-2), interleukin-3 (IL-3), interleukin-6 (IL-6), interleukin-7 (IL-7), interleukin-15 (IL-15), FMS-like tyrosine kinase 3 ligand (FLT3L), leukemia inhibitory factor (LIF), insulin-like growth factor (IGF), and insulin, and combinations thereof. Suitable cytokines for the maintenance and proliferation of hematopoietic progenitors and myeloid commited cells are for instance SCF, TPO, FLT3L, G-CSF, IL-3, IL-6 and combinations thereof; a preferred cytokine combination for the maintenance and proliferation of hematopoietic progenitors and myeloid commited cells being SCF, TPO, FLT3L, G-CSF and IL-3.

In a preferred embodiment of the first aspect of the invention, the metabolic disease is pyruvate kinase deficiency (PKD).

In another preferred embodiment of the first aspect of the invention, the metabolic disease is pyruvate kinase deficiency (PKD), and the gene editing is performed via a knock-in strategy by using a therapeutic matrix comprising a partial codon-optimized (cDNA) RPK gene covering exons 3 to 11 preceded by a splice acceptor signal, wherein these elements are flanked by two homology arms matching sequences in the target locus of the PKLR gene, and wherein this matrix is introduced by homologous recombination in the target locus of the PKLR gene. Preferably, the gene editing is performed via a knock-in strategy by using a therapeutic matrix comprising a partial codon-optimized (cDNA) RPK gene covering exons 3 to 11 preceded by a splice acceptor signal, wherein these elements are flanked by two homology arms matching sequences in the second intron of the PKLR gene, and wherein this matrix is introduced by homologous recombination in the second intron of the PKLR locus. More preferably, the therapeutic matrix further comprises a positive-negative selection cassette preferably comprising a puromycin (Puro) resistance/thymidine (TK) fusion gene driven by a phosphoglycerate kinase promoter downstream of the partial codon-optimized (cDNA) PKLR gene.

A second aspect of the invention, refers to a process to promote the maintenance and proliferation of hematopoietic progenitors and myeloid-committed cells, which comprises culturing peripheral blood mononuclear cells isolated from a mammalian subject, preferably from a human subject, and expanding these cells in the presence of SCF, TPO, FLT3L, granulocyte colony-stimulating factor (G-CSF) and IL-3, preferably for at least 4 days, and optionally collecting these cells.

A third aspect of the invention, refers to a process of producing induced pluripotent stem cells or a cell population comprising induced pluripotent stem cells, derived from peripheral blood mononuclear cells, comprising the following steps:
a. Culturing peripheral blood mononuclear cells isolated from a mammalian subject, preferably from a human subject, and expanding these cells in the presence of SCF, TPO, FLT3L, granulocyte colony-stimulating factor (G-CSF) and IL-3 to promote the maintenance and proliferation of hematopoietic progenitors and myeloid-committed cells, preferably for at least 4 days; and
b. Reprogramming the cells obtained from step a) above, by preferably using a transduction protocol using the Sendai viral vector platform (SeV) encoding the following four reprograming factors: OCT3/4, KLF4, SOX2 and c-MYC, and maintaning these cells preferably from 3 to 6 days, preferably in the same medium; and
c. optionally, collecting the cells.

In a preferred embodiment of the third aspect of the invention, the peripheral blood mononuclear cells are isolated from a subject suffering from a metabolic disease affecting the erythroid lineage; preferably, suffering from pyruvate kinase deficiency (PKD).

In another preferred embodiment of the third aspect of the invention, the peripheral blood mononuclear cells are isolated from a subject suffering from a metabolic disease affecting the erythroid lineage, and the process further comprises the further step of:
d. correcting the mutation or mutations in the gene causing the metabolic disease present in the induced pluripotent stem cells, by gene-editing via a knock-in strategy where a partial cDNA is inserted in a locus of the target gene to express a chimeric mRNA formed by endogenous first exons and partial cDNA under the endogenous promoter control, wherein preferably nucleases are used to promote homologous recombination (HR); and
e. optionally, collecting the knock-in cells.

In another preferred embodiment of the third aspect of the invention, the peripheral blood mononuclear cells are isolated from a subject suffering from pyruvate kinase deficiency (PKD), and the process further comprises the further step of:
d. correcting the mutation or mutations in the PKLR gene present in the induced pluripotent stem cells, by gene-editing the PKLR gene via a knock-in strategy by using a therapeutic matrix comprising a partial codon-optimized (cDNA) RPK gene covering exons 3 to 11 preceded by a splice acceptor signal, wherein these elements are flanked by two homology arms matching sequences in the target locus of the PKLR gene and wherein this matrix is introduced by homologous recombination in the target locus of the PKLR gene, wherein preferably nucleases are used to promote HR; and
e. optionally, collecting the knock-in cells.

In another preferred embodiment of the third aspect of the invention, the peripheral blood mononuclear cells are isolated from a subject suffering from pyruvate kinase deficiency (PKD), and the process further comprises the further step of:
d. correcting the mutation or mutations in the PKLR gene present in the induced pluripotent stem cells, by gene-editing the PKLR gene via a knock-in strategy by using a therapeutic matrix comprising a partial codon-optimized (cDNA) RPK gene covering exons 3 to 11 preceded by a splice acceptor signal, wherein these elements are flanked by two homology arms matching sequences in the second intron of the PKLR gene and wherein this matrix is introduced by homologous recombination in the second intron of the PKLR gene, wherein preferably nucleases are used to promote HR; and
e. optionally, collecting the knock-in cells.

Various nucleases for genome editing are well known in the art, these include: TALENs (transcription activator-like effector nucleases), CRISPR/Cas (clustered regulatory interspaced short palindromic repeats), zinc finger nucleases and meganucleases (e.g., the LAGLIDADG family of homing endonucleases). For a review, see for instance: Lopez-Manzaneda S. 2016.

In a preferred embodiment of the third aspect of the invention, said nuclease is a PKLR transcription activator-like effector nuclease (TALEN), preferably wherein said nuclease is a PKLR TALEN which comprises two subunits defined by SEQ ID NO:1 and SEQ ID NO:2.

(LEFT SUBUNIT PKLR TALEN)
SEQ ID NO: 1
ATGGGCGATCCTAAAAAGAAACGTAAGGTCATCGATTACCCATACGATGT
TCCAGATTACGCTATCGATATCGCCGATCTACGCACGCTCGGCTACAGCC
AGCAGCAACAGGAGAAGATCAAACCGAAGGTTCGTTCGACAGTGGCGCAG
CACCACGAGGCACTGGTCGGCCACGGGTTTACACACGCGCACATCGTTGC
GTTAAGCCAACACCCGGCAGCGTTAGGGACCGTCGCTGTCAAGTATCAGG
ACATGATCGCAGCGTTGCCAGAGGCGACACACGAAGCGATCGTTGGCGTC
GGCAAACAGTGGTCCGGCGCACGCGCTCTGGAGGCCTTGCTCACGGTGGC
GGGAGAGTTGAGAGGTCCACCGTTACAGTTGGACACAGGCCAACTTCTCA
AGATTGCAAAACGTGGCGGCGTGACCGCAGTGGAGGCAGTGCATGCATGG
CGCAATGCACTGACGGGTGCCCCGCTCAACTTGACCCCCCAGCAGGTGGT
GGCCATCGCCAGCAATAATGGTGGCAAGCAGGCGCTGGAGACGGTCCAGC
GGCTGTTGCCGGTGCTGTGCCAGGCCCACGGCTTGACCCCGGAGCAGGTG
GTGGCCATCGCCAGCAATATTGGTGGCAAGCAGGCGCTGGAGACGGTGCA
GGCGCTGTTGCCGGTGCTGTGCCAGGCCCACGGCTTGACCCCCCAGCAGG
TGGTGGCCATCGCCAGCAATGGCGGTGGCAAGCAGGCGCTGGAGACGGTC
CAGCGGCTGTTGCCGGTGCTGTGCCAGGCCCACGGCTTGACCCCGGAGCA
GGTGGTGGCCATCGCCAGCCACGATGGCGGCAAGCAGGCGCTGGAGACGG
TCCAGCGGCTGTTGCCGGTGCTGTGCCAGGCCCACGGCTTGACCCCCCAG
CAGGTGGTGGCCATCGCCAGCAATAATGGTGGCAAGCAGGCGCTGGAGAC
GGTCCAGCGGCTGTTGCCGGTGCTGTGCCAGGCCCACGGCTTGACCCCGG
AGCAGGTGGTGGCCATCGCCAGCAATATTGGTGGCAAGCAGGCGCTGGAG
ACGGTGCAGGCGCTGTTGCCGGTGCTGTGCCAGGCCCACGGCTTGACCCC
CCAGCAGGTGGTGGCCATCGCCAGCAATAATGGTGGCAAGCAGGCGCTGG
AGACGGTCCAGCGGCTGTTGCCGGTGCTGTGCCAGGCCCACGGCTTGACC
CCGGAGCAGGTGGTGGCCATCGCCAGCCACGATGGCGGCAAGCAGGCGCT
GGAGACGGTCCAGCGGCTGTTGCCGGTGCTGTGCCAGGCCCACGGCTTGA
CCCCGGAGCAGGTGGTGGCCATCGCCAGCCACGATGGCGGCAAGCAGGCG
CTGGAGACGGTCCAGCGGCTGTTGCCGGTGCTGTGCCAGGCCCACGGCTT
GACCCCGGAGCAGGTGGTGGCCATCGCCAGCAATATTGGTGGCAAGCAGG
CGCTGGAGACGGTGCAGGCGCTGTTGCCGGTGCTGTGCCAGGCCCACGGC
TTGACCCCGGAGCAGGTGGTGGCCATCGCCAGCCACGATGGCGGCAAGCA
GGCGCTGGAGACGGTCCAGCGGCTGTTGCCGGTGCTGTGCCAGGCCCACG
GCTTGACCCCCCAGCAGGTGGTGGCCATCGCCAGCAATGGCGGTGGCAAG
CAGGCGCTGGAGACGGTCCAGCGGCTGTTGCCGGTGCTGTGCCAGGCCCA
CGGCTTGACCCCCCAGCAGGTGGTGGCCATCGCCAGCAATAATGGTGGCA
AGCAGGCGCTGGAGACGGTCCAGCGGCTGTTGCCGGTGCTGTGCCAGGCC
CACGGCTTGACCCCCCAGCAGGTGGTGGCCATCGCCAGCAATGGCGGTGG
CAAGCAGGCGCTGGAGACGGTCCAGCGGCTGTTGCCGGTGCTGTGCCAGG
CCCACGGCTTGACCCCGGAGCAGGTGGTGGCCATCGCCAGCAATATTGGT
GGCAAGCAGGCGCTGGAGACGGTGCAGGCGCTGTTGCCGGTGCTGTGCCA
GGCCCACGGCTTGACCCCTCAGCAGGTGGTGGCCATCGCCAGCAATGGCG
GCGGCAGGCCGGCGCTGGAGAGCATTGTTGCCCAGTTATCTCGCCCTGAT
CCGGCGTTGGCCGCGTTGACCAACGACCACCTCGTCGCCTTGGCCTGCCT
CGGCGGGCGTCCTGCGCTGGATGCAGTGAAAAAGGGATTGGGGGATCCTA
TCAGCCGTTCCCAGCTGGTGAAGTCCGAGCTGGAGGAGAAGAAATCCGAG
TTGAGGCACAAGCTGAAGTACGTGCCCCACGAGTACATCGAGCTGATCGA
GATCGCCCGGAACAGCACCCAGGACCGTATCCTGGAGATGAAGGTGATGG
AGTTCTTCATGAAGGTGTACGGCTACAGGGGCAAGCACCTGGGCGGCTCC
AGGAAGCCCGACGGCGCCATCTACACCGTGGGCTCCCCCATCGACTACGG
CGTGATCGTGGACACCAAGGCCTACTCCGGCGGCTACAACCTGCCCATCG
GCCAGGCCGACGAAATGCAGAGGTACGTGGAGGAGAACCAGACCAGGAAC
AAGCACATCAACCCCAACGAGTGGTGGAAGGTGTACCCCTCCAGCGTGAC
CGAGTTCAAGTTCCTGTTCGTGTCCGGCCACTTCAAGGGCAACTACAAGG
CCCAGCTGACCAGGCTGAACCACATCACCAACTGCAACGGCGCCGTGCTG
TCCGTGGAGGAGCTCCTGATCGGCGGCGAGATGATCAAGGCCGGCACCCT
GACCCTGGAGGAGGTGAGGAGGAAGTTCAACAACGGCGAGATCAACTTCG
CGGCCGACTGATAA
(RIGHT SUBUNIT PKLR TALEN)
SEQ ID NO: 2
ATGGGCGATCCTAAAAAGAAACGTAAGGTCATCGATAAGGAGACCGCCGC
TGCCAAGTTCGAGAGACAGCACATGGACAGCATCGATATCGCCGATCTAC
GCACGCTCGGCTACAGCCAGCAGCAACAGGAGAAGATCAAACCGAAGGTT
CGTTCGACAGTGGCGCAGCACCACGAGGCACTGGTCGGCCACGGGTTTAC
ACACGCGCACATCGTTGCGTTAAGCCAACACCCGGCAGCGTTAGGGACCG
TCGCTGTCAAGTATCAGGACATGATCGCAGCGTTGCCAGAGGCGACACAC
GAAGCGATCGTTGGCGTCGGCAAACAGTGGTCCGGCGCACGCGCTCTGGA
GGCCTTGCTCACGGTGGCGGGAGAGTTGAGAGGTCCACCGTTACAGTTGG
ACACAGGCCAACTTCTCAAGATTGCAAAACGTGGCGGCGTGACCGCAGTG
GAGGCAGTGCATGCATGGCGCAATGCACTGACGGGTGCCCCGCTCAACTT
GACCCCGGAGCAGGTGGTGGCCATCGCCAGCCACGATGGCGGCAAGCAGG
CGCTGGAGACGGTCCAGCGGCTGTTGCCGGTGCTGTGCCAGGCCCACGGC
TTGACCCCCCAGCAGGTGGTGGCCATCGCCAGCAATGGCGGTGGCAAGCA
GGCGCTGGAGACGGTCCAGCGGCTGTTGCCGGTGCTGTGCCAGGCCCACG
GCTTGACCCCGGAGCAGGTGGTGGCCATCGCCAGCCACGATGGCGGCAAG
CAGGCGCTGGAGACGGTCCAGCGGCTGTTGCCGGTGCTGTGCCAGGCCCA
CGGCTTGACCCCCCAGCAGGTGGTGGCCATCGCCAGCAATGGCGGTGGCA
AGCAGGCGCTGGAGACGGTCCAGCGGCTGTTGCCGGTGCTGTGCCAGGCC
CACGGCTTGACCCCGGAGCAGGTGGTGGCCATCGCCAGCAATATTGGTGG
CAAGCAGGCGCTGGAGACGGTGCAGGCGCTGTTGCCGGTGCTGTGCCAGG
CCCACGGCTTGACCCCCCAGCAGGTGGTGGCCATCGCCAGCAATAATGGT
GGCAAGCAGGCGCTGGAGACGGTCCAGCGGCTGTTGCCGGTGCTGTGCCA
GGCCCACGGCTTGACCCCCCAGCAGGTGGTGGCCATCGCCAGCAATAATG
GTGGCAAGCAGGCGCTGGAGACGGTCCAGCGGCTGTTGCCGGTGCTGTGC
CAGGCCCACGGCTTGACCCCCCAGCAGGTGGTGGCCATCGCCAGCAATAA
TGGTGGCAAGCAGGCGCTGGAGACGGTCCAGCGGCTGTTGCCGGTGCTGT
GCCAGGCCCACGGCTTGACCCCCCAGCAGGTGGTGGCCATCGCCAGCAAT
GGCGGTGGCAAGCAGGCGCTGGAGACGGTCCAGCGGCTGTTGCCGGTGCT
GTGCCAGGCCCACGGCTTGACCCCGGAGCAGGTGGTGGCCATCGCCAGCC
ACGATGGCGGCAAGCAGGCGCTGGAGACGGTCCAGCGGCTGTTGCCGGTG
CTGTGCCAGGCCCACGGCTTGACCCCCCAGCAGGTGGTGGCCATCGCCAG
CAATGGCGGTGGCAAGCAGGCGCTGGAGACGGTCCAGCGGCTGTTGCCGG
TGCTGTGCCAGGCCCACGGCTTGACCCCGGAGCAGGTGGTGGCCATCGCC
AGCCACGATGGCGGCAAGCAGGCGCTGGAGACGGTCCAGCGGCTGTTGCC
GGTGCTGTGCCAGGCCCACGGCTTGACCCCCCAGCAGGTGGTGGCCATCG
CCAGCAATAATGGTGGCAAGCAGGCGCTGGAGACGGTCCAGCGGCTGTTG
CCGGTGCTGTGCCAGGCCCACGGCTTGACCCCCCAGCAGGTGGTGGCCAT
CGCCAGCAATGGCGGTGGCAAGCAGGCGCTGGAGACGGTCCAGCGGCTGT
TGCCGGTGCTGTGCCAGGCCCACGGCTTGACCCCGGAGCAGGTGGTGGCC
ATCGCCAGCCACGATGGCGGCAAGCAGGCGCTGGAGACGGTCCAGCGGCT
GTTGCCGGTGCTGTGCCAGGCCCACGGCTTGACCCCTCAGCAGGTGGTGG
CCATCGCCAGCAATGGCGGCGGCAGGCCGGCGCTGGAGAGCATTGTTGCC
CAGTTATCTCGCCCTGATCCGGCGTTGGCCGCGTTGACCAACGACCACCT
CGTCGCCTTGGCCTGCCTCGGCGGGCGTCCTGCGCTGGATGCAGTGAAAA
AGGGATTGGGGGATCCTATCAGCCGTTCCCAGCTGGTGAAGTCCGAGCTG
GAGGAGAAGAAATCCGAGTTGAGGCACAAGCTGAAGTACGTGCCCCACGA
GTACATCGAGCTGATCGAGATCGCCCGGAACAGCACCCAGGACCGTATCC
TGGAGATGAAGGTGATGGAGTTCTTCATGAAGGTGTACGGCTACAGGGGC
AAGCACCTGGGCGGCTCCAGGAAGCCCGACGGCGCCATCTACACCGTGGG
CTCCCCCATCGACTACGGCGTGATCGTGGACACCAAGGCCTACTCCGGCG
GCTACAACCTGCCCATCGGCCAGGCCGACGAAATGCAGAGGTACGTGGAG
GAGAACCAGACCAGGAACAAGCACATCAACCCCAACGAGTGGTGGAAGGT
GTACCCCTCCAGCGTGACCGAGTTCAAGTTCCTGTTCGTGTCCGGCCACT
TCAAGGGCAACTACAAGGCCCAGCTGACCAGGCTGAACCACATCACCAAC
TGCAACGGCGCCGTGCTGTCCGTGGAGGAGCTCCTGATCGGCGGCGAGAT
GATCAAGGCCGGCACCCTGACCCTGGAGGAGGTGAGGAGGAAGTTCAACA
ACGGCGAGATCAACTTCGCGGCCGACTGATAA

In another preferred embodiment of the third aspect of the invention, said nuclease is used as mRNA, preferably with 5′ and/or 3′ modifications, more preferably wherein 5′UTR VEEV (SEQ ID NO: 3: ACTAGCGCTATGGGCGGCGCATGAGAGAAGCCCAGACCAATTACCTACCCAAA) has been added in the 5′ end and/or 3′UTR b-Globin (SEQ ID NO:4 CTCGAGATTTCTATTAAAGGTTCCTTTGTTCCCTAAGTCCAACTACTAAACTGGGGGATATT ATGAAGGGCCTTGAGCATCGTCGAC) has been added in the 3′ end.

Introduction of the therapeutic matrix and optionally said nucleases into the host cells in a process according to the third aspect of the present invention, may be carried out by transformation or transfection methods well known in the art such as nucleofection, lipofection etc. See, e.g., Green & Sambrook, Molecular Cloning: A Laboratory Manual, Fourth Edition. Cold Spring Harbor, N.Y.: Cold Spring Harbor Laboratory Press, 2012.

A fourth aspect of the invention refers to the induced pluripotent stem cells obtained or obtainable by the process of the third aspect of the invention or of any of its preferred embodiments.

A fifth aspect of the invention refers to the induced pluripotent stem cells according to the first aspect of the invention or according to the fourth aspect of the invention, for its use in therapy.

A sixth aspect of the invention refers to the induced pluripotent stem cells according to the first aspect of the invention or according to the fourth aspect of the invention, for its use in the treatment of a metabolic disease affecting the erythroid lineage; preferably, for its use in the treatment of pyruvate kinase deficiency (PKD).

A seventh aspect of the invention refers to a therapeutic matrix comprising a partial codon-optimized (cDNA) RPK gene covering exons 3 to 11 preceded by a splice acceptor signal, wherein these elements are flanked by two homology arms matching sequences in a target locus of the PKLR gene, and wherein this matrix is capable of introducing itself by homologous recombination in the target locus of the PKLR gene.

In a preferred embodiment, said therapeutic matrix comprises a partial codon-optimized (cDNA) RPK gene covering exons 3 to 11 (SEQ ID NO:5), fused to a tag and preceded by a splice acceptor signal (SEQ ID NO:7: CTCTTCCTCCCACAG).

(coRPK E3-E11)
SEQ ID NO: 5
GCCCTGCCAGCAGAAGCGTGGAGCGGCTGAAAGAGATGATCAAGGCCGGC
ATGAATATCGCCCGGCTGAACTTCTCCCACGGCAGCCACGAGTACCACGC
AGAGAGCATTGCCAACGTCCGGGAGGCCGTGGAGAGCTTTGCCGGCAGCC
CCCTGAGCTACAGACCCGTGGCCATTGCCCTGGACACCAAGGGCCCCGAG
ATCAGAACAGGAATTCTGCAGGGAGGGCCTGAGAGCGAGGTGGAGCTGGT
GAAGGGCAGCCAAGTGCTGGTGACCGTGGACCCCGCCTTCAGAACCAGAG
GCAACGCCAACACAGTGTGGGTGGACTACCCCAACATCGTGCGGGTGGTG
CCTGTGGGCGGCAGAATCTACATCGACGACGGCCTGATCAGCCTGGTGGT
GCAGAAGATCGGACCTGAGGGCCTGGTGACCCAGGTCGAGAATGGCGGCG
TGCTGGGCAGCAGAAAGGGCGTGAATCTGCCAGGCGCCCAGGTGGACCTG
CCTGGCCTGTCTGAGCAGGACGTGAGAGACCTGAGATTTGGCGTGGAGCA
CGGCGTGGACATCGTGTTCGCCAGCTTCGTGCGGAAGGCCTCTGATGTGG
CCGCCGTGAGAGCCGCTCTGGGCCCTGAAGGCCACGGCATCAAGATCATC
AGCAAGATCGAGAACCACGAGGGCGTGAAGCGGTTCGACGAGATCCTGGA
AGTGTCCGACGGCATCATGGTGGCCAGAGGCGACCTGGGCATCGAGATCC
CCGCCGAGAAGGTGTTCCTGGCCCAGAAAATGATGATCGGACGGTGCAAC
CTGGCCGGCAAACCTGTGGTGTGCGCCACCCAGATGCTGGAAAGCATGAT
CACCAAGCCCAGACCCACCAGAGCCGAGACAAGCGACGTGGCCAACGCCG
TGCTGGATGGCGCTGACTGCATCATGCTGTCCGGCGAGACAGCCAAGGGC
AACTTCCCCGTGGAGGCCGTGAAGATGCAGCACGCCATTGCCAGAGAAGC
CGAGGCCGCCGTGTACCACCGGCAGCTGTTCGAGGAACTGCGGAGAGCCG
CCCCTCTGAGCAGAGATCCCACCGAAGTGACCGCCATCGGAGCCGTGGAA
GCCGCCTTCAAGTGCTGCGCCGCTGCAATCATCGTGCTGACCACCACAGG
CAGAAGCGCCCAGCTGCTGTCCAGATACAGACCCAGAGCCGCCGTGATCG
CCGTGACAAGATCCGCCCAGGCCGCTAGACAGGTCCACCTGTGCAGAGGC
GTGTTCCCCCTGCTGTACCGGGAGCCTCCCGAGGCCATCTGGGCCGACGA
CGTGGACAGACGGGTGCAGTTCGGCATCGAGAGCGGCAAGCTGCGGGGCT
TCCTGAGAGTGGGCGACCTGGTGATCGTGGTGACAGGCTGGCGGCCTGGC
AGCGGCTACACCAACATCATGAGGGTGCTGTCCATCAGC

Different tags well known in the art may be used. These include but are not limited to 3×FLAG, Poly-Arg-tag, Poly-His-tag, Strep-tag II, c-myc-tag, S-tag, HAT-tag, Calmodulin-binding peptide-flag, Cellulose-binding domains-tag, SBP-tag, Chitin-binding domain-tag, Glutathione 5-transferase-tag or Maltose-binding protein-tag. Preferably, said tag is a FLAG tag (SEQ ID NO: 6: GACTACAAAGACGATGACGATAAATGA)

In a more preferred embodiment, the therapeutic matrix further comprises a positive-negative selection cassette. Different selection markers can be used, such as resistance gene to antibiotics neomycin phosphotransferase (neo), dihydrofolate reductase (DHFR), or glutamine synthetase, surface gene (CD4 or truncated NGFR), luciferase or fluorescent proteins (eGFP, mCherry, mTomato, etc)

Preferably said positive-negative selection cassette is a puromycin (Puro) resistance/thymidine (TK) fusion gene driven by a phosphoglycerate kinase (PGK) promoter downstream of the partial codon-optimized (cDNA) PKLR gene. Instead of PGK other promoters may also be used such as Elongation Factor-1 alpha (EFlalpha), spleen focus forming virus (SSFV), quimeric cytomegalovirus enhancer plus chiken beta actin promoter, first exon and first intron plus splicing acceptor of the rabbit beta globin gene (CAG), cytomegalovirus (CMV) or any other ubiquotous or hematopoietic specific promoter

Preferably, said positive-negative selection cassette contains a puromycin (Puro) resistance/thymidine kinase (TK) fusion gene driven by mouse phosphoglycerate kinase (mPGK) promoter (SEQ ID NO:8) located downstream of the partial (cDNA) RPK.

SEQ ID 8: mPGK-Puro/TK
CCGGGTAGGGGAGGCGCTTTTCCCAAGGCAGTCTGGAGCATGCGCTTTAG
CAGCCCCGCTGGGCACTTGGCGCTACACAAGTGGCCTCTGGCCTCGCACA
CATTCCACATCCACCGGTAGGCGCCAACCGGCTCCGTTCTTTGGTGGCCC
CTTCGCGCCACCTTCTACTCCTCCCCTAGTCAGGAAGTTCCCCCCCGCCC
CGCAGCTCGCGTCGTGCAGGACGTGACAAATGGAAGTAGCACGTCTCACT
AGTCTCGTGCAGATGGACAGCACCGCTGAGCAATGGAAGCGGGTAGGCCT
TTGGGGCAGCGGCCAATAGCAGCTTTGCTCCTTCGCTTTCTGGGCTCAGA
GGCTGGGAAGGGGTGGGTCCGGGGGCGGGCTCAGGGGCGGGCTCAGGGGC
GGGGCGGGCGCCCGAAGGTCCTCCGGAGGCCCGGCATTCTGCACGCTTCA
AAAGCGCACGTCTGCCGCGCTGTTCTCCTCTTCCTCATCTCCGGGCCTTT
CGACCGATCATCAAGCTTGATCCTCATGACCGAGTACAAGCCCACGGTGC
GCCTCGCCACCCGCGACGACGTCCCCAGGGCCGTACGCACCCTCGCCGCC
GCGTTCGCCGACTACCCCGCCACGCGCCACACCGTCGATCCGGACCGCCA
CATCGAGCGGGTCACCGAGCTGCAAGAACTCTTCCTCACGCGCGTCGGGC
TCGACATCGGCAAGGTGTGGGTCGCGGACGACGGCGCCGCGGTGGCGGTC
TGGACCACGCCGGAGAGCGTCGAAGCGGGGGCGGTGTTCGCCGAGATCGG
CCCGCGCATGGCCGAGTTGAGCGGTTCCCGGCTGGCCGCGCAGCAACAGA
TGGAAGGCCTCCTGGCGCCGCACCGGCCCAAGGAGCCCGCGTGGTTCCTG
GCCACCGTCGGCGTCTCGCCCGACCACCAGGGCAAGGGTCTGGGCAGCGC
CGTCGTGCTCCCCGGAGTGGAGGCGGCCGAGCGCGCCGGGGTGCCCGCCT
TCCTGGAGACCTCCGCGCCCCGCAACCTCCCCTTCTACGAGCGGCTCGGC
TTCACCGTCACCGCCGACGTCGAGGTGCCCGAAGGACCGCGCACCTGGTG
CATGACCCGCAAGCCCGGTGCCGGATCCATGCCCACGCTACTGCGGGTTT
ATATAGACGGTCCCCACGGGATGGGGAAAACCACCACCACGCAACTGCTG
GTGGCCCTGGGTTCGCGCGACGATATCGTCTACGTACCCGAGCCGATGAC
TTACTGGCGGGTGCTGGGGGCTTCCGAGACAATCGCGAACATCTACACCA
CACAACACCGCCTCGACCAGGGTGAGATATCGGCCGGGGACGCGGCGGTG
GTAATGACAAGCGCCCAGATAACAATGGGCATGCCTTATGCCGTGACCGA
CGCCGTTCTGGCTCCTCATATCGGGGGGGAGGCTGGGAGCTCACATGCCC
CGCCCCCGGCCCTCACCCTCATCTTCGACCGCCATCCCATCGCCGCCCTC
CTGTGCTACCCGGCCGCGCGGTACCTTATGGGCAGCATGACCCCCCAGGC
CGTGCTGGCGTTCGTGGCCCTCATCCCGCCGACCTTGCCCGGCACCAACA
TCGTGCTTGGGGCCCTTCCGGAGGACAGACACATCGACCGCCTGGCCAAA
CGCCAGCGCCCCGGCGAGCGGCTGGACCTGGCTATGCTGGCTGCGATTCG
CCGCGTTTACGGGCTACTTGCCAATACGGTGCGGTATCTGCAGTGCGGCG
GGTCGTGGCGGGAGGACTGGGGACAGCTTTCGGGGACGGCCGTGCCGCCC
CAGGGTGCCGAGCCCCAGAGCAACGCGGGCCCACGACCCCATATCGGGGA
CACGTTATTTACCCTGTTTCGGGCCCCCGAGTTGCTGGCCCCCAACGGCG
ACCTGTATAACGTGTTTGCCTGGGCCTTGGACGTCTTGGCCAAACGCCTC
CGTTCCATGCACGTCTTTATCCTGGATTACGACCAATCGCCCGCCGGCTG
CCGGGACGCCCTGCTGCAACTTACCTCCGGGATGGTCCAGACCCACGTCA
CCACCCCCGGCTCCATACCGACGATATGCGACCTGGCGCGCACGTTTGCC
CGGGAGATGGGGGAGGCTAACTGAGCTCTAGAGCGGCCAGTGTCGCGGTA
TCGATGAGCTAGAGCTCGCTGATCAGCCTCGACTGTGCCTTCTAGTTGCC
AGCCATCTGTTGTTTGCCCCTCCCCCGTGCCTTCCTTGACCCTGGAAGGT
GCCACTCCC

Preferably, these elements are flanked by two homology arms (SEQ ID NO:9 and 10) matching sequences in the second intron of the PKLR gene (FIG. 2A).

(Left Homology Arm)
SEQ ID NO: 9
GCGGCGGGCCAGTGTGGCCCAACTGACCCAGGAGCTGGGCACTGCCTTCT
TCCAGCAGCAGCAGCTGCCAGCTGCTATGGCAGACACCTTCCTGGAACAC
CTCTGCCTACTGGACATTGACTCCGAGCCCGTGGCTGCTCGCAGTACCAG
CATCATTGCCACCATCGGTAAGCACTCCCATCCCCCTGCAGCCACACAGG
GCCTATTGGTATTTCTTGAGGTGCTTCTTCATCTTTTGTCTCCTTTGAGA
CTTCTCCATGTTTGACACAGTCATTCATTTAACAAAAATTTGTTGAGCAT
ATAGTAGACAAGATTTTGGGCCCTGGGAGTAGATCAGTGAAAAAAACAGA
CAAAAATCCCTACCCTTGGGGAGCTGACAGTCTAGCTGAGTATGACAATA
AATAGTAAGCACAATAAATTATTTAAAATAAGTAAATTATTTATTCCGTT
AGAAAGTGAGGCCGGGCATGGTGGCTCATGCCTGTAATCGCAGCATGTTG
GGAGGCCCAGGTGGGCAGATCACTTGAGGTCAGGAGTTCGAGACTAGCCT
GACCAACATGGAGAAACCCCGTCTCTACTAAAAATACAAAATTAGCCGGG
CATGGTGGTGCGTGCCTGCAATCCCAGCTACTCAGGAGGCTGAGGCAGGA
GAATCGCTTGAACCCAGGAGGCGGAGACTGTGGTGAGCCGAGATCACACC
ATTGCATTCCAGCCTGGGCAACAGGAGAAAAACTCCATCTCACAAAAAAA
AAAAAAAAAAAAAAAAAAAAAAAGTGGGCTGGGCTCAGTGGCTCATGCCT
GTAATCCCAGCACTTTAGGAGGCCAAGGTTGGCAGATCGCTTGAGCCCAG
GAGTTTGAGACCAGTCTGGGTAAATGGCAAAACCCATCTCTACAAAAAAT
ACAAAACTTAGTTGAGTGTGGTGGTGCATGCCTGTAGTCCCAGCTACTCA
GGAGGCTGAGGTGGGAGGATCACTTAAGCCCAG
(Right Homology Arm)
SEQ ID NO: 10
GAGAGAAAGAAAGAAAGAAGGAAAGAAAGAAAGAAAGAGAGAGAGAAAGA
AGGAAGGAAGGAAGGAGGGAGGGAGGGAGGGAAGGAAGGAAGGAAAGAAA
GCAAGCAGGCAAGAAAGAAAGAAAGAAAAGAAAGAAGGAAGGAAGGAAGG
AAGGAAAGAAAGAAAGAAAGAGAAAGAAAGAAAGAAAGAAAGAAAGAAAG
AAAGAAAGAAAGAAAGAAAGAAAGAAAGAAAGAAAGAAAGAAAGAAAGAA
AGGAGTGAAAGTTGGCCGGGCATGGTGGCTCTTGCCTATAATCCCAGCAC
TTTGGGAGGCTGAGGCAGGTGGATCACCTGAGGTCAGGGGTCCGAGACCA
GCCTGGCTAATGTGGTGAAACTCTGTTTCTACTAAAAATACAAAAAATTA
GCCAGGCATGGTGGCATGTGCCTATAATCCCAGCTACTCGGGAGGCTGAG
GCAGGGGAATCGCTTGAACCCGGGAGACAGAGATTGCAGTGAGCCAAGAT
CACGCCATTGCACTCCAGTTTGGGCAACAAGAGCGAAACTCTGTTTGTTT
GTTTGTTTGTTTTTAAAAAAAGAAAAAAAAGCTGGGCGCGGTGGCTCACG
CCTGTAATCCCAGCACTTTGGGAGGCCGAGGCGGGCGGATCACCTGAGGT
CAGGAGTTCGAGACCAGCCTCAACATGGAGAAACCCCGTCTCTACTAAAA
ATACAAAAAATTATCCGGGCATGGTGGTGCATGCCTGTAATCCCAGCTAC
TCAGGAGGCTAAGGCAGGAGAATTGCTTGAACCTGGGAGGCGGAGGTTGC
GGTGAGCCAAGATCGTGCCATTGCACCCCAGCCTGGGCAACAAGAGCGAA
ACTCCGTCTCAAAAAAAAAAAAGGCCAGGCGTGGTGTTTCATGCCTGTAA
TCCCAGCACTTTGGGAGGCCGAGGCAGACTGATCACGAGGTCAAGAGATC
GATACCATCCTGGCCAACATG

In order to increase the efficiency of gene editing, the inventors developed a PKLR-specific TALEN targeting a specific genomic sequence in the second intron (SEQ ID NO:11) flanked by the homology arms:

TGATCGAGCCACTGTACTCCAGCCTAGGTGACAGACGAGACCCTAGAGA
(left and right PKLR TALEN recognition site are
underlined).

Accordingly, the invention also provides a specifically designed PKLR transcription activator-like effector nuclease (TALEN). More specifically, it comprises two PKLR TALEN subunits. The left subunit of PKLR TALEN is defined by SEQ ID NO:1 and the right subunit of PKLR TALEN is defined by SEQ ID NO:2.

An eighth aspect of the invention, refers to the ex vivo, or in vitro, use of the therapeutic matrix of the fourth aspect of the invention, for correcting, by gene-editing via a knock-in strategy, the mutation or mutations in the PKLR gene in induced pluripotent stem cells derived from peripheral blood mononuclear cells of the erythroid lineage isolated from a subject suffering from pyruvate kinase deficiency (PKD).

A ninth aspect of the invention refers to a Sendai viral vector platform (SeV) encoding the following four reprograming factors: OCT3/4, KLF4, SOX2 and c-MYC.

A tenth aspect of the invention, refers to the ex vivo, or in vitro, use of the Sendai viral vector platform of the ninth aspect of the invention, for reprogramming peripheral blood mononuclear cells of the erythroid lineage isolated from a subject suffering from a metabolic disease affecting the erythroid lineage. Preferably, for reprogramming peripheral blood mononuclear cells of the erythroid lineage isolated from a subject suffering from pyruvate kinase deficiency (PKD).

An eleventh aspect of the invention, refers to the ex vivo, or in vitro, use of a composition, preferably a cell media, which comprises SCF, TPO, FLT3L, granulocyte colony-stimulating factor (G-CSF) and IL-3 for promoting the maintenance and proliferation of hematopoietic progenitors and myeloid-committed cells.

A twelfth aspect, refers to a cell population comprising peripheral blood mononuclear cells of the erythroid lineage derived from inducing the erythroid differentiation of the induced pluripotent stem cells of any of the precedent aspects of the invention. Preferably, these cells are use in the treatment of a metabolic disease affecting the erythroid lineage, more preferably for the treatment of pyruvate kinase deficiency (PKD).

A thirteenth aspect of the invention refers to the process of the third aspect of the invention or of any of its preferred embodiments, which further comprises the step of inducing the erythroid differentiation of the induced pluripotent stem cells and optionally collecting the peripheral blood mononuclear cells of the erythroid lineage resulting from said differentiation process.

The following examples merely illustrate but do not limit the present invention.

### EXAMPLES

### Example 1

### Generation of Integration-Free Specific iPSCs Derived from the Peripheral Blood of PKD Patients

First, to evaluate the potential use of PB-MNCs as a cell source to be reprogrammed to iPSCs by the non-integrative SeV, we analyzed the susceptibility of these cells to SeV. PB-MNCs were expanded in the presence of specific cytokines (stem cell factor [SCF], thrombopoietin [TPO], FLT3L, granulocyte colony-stimulating factor [G-CSF], and IL-3) to promote the maintenance and proliferation of hematopoietic progenitors and myeloid-committed cells for 4 days. Cells were then infected with a SeV encoding for the Azami green fluorescent marker. Five days later, the transduction of hematopoietic progenitor (CD34+), myeloid (CD14+/CD15+), and lymphoid T (CD3+) and B (CD19+) cells was evaluated by flow cytometry. Although the majority of cells in the culture expressed Tor B lymphoid markers, a reduced proportion of them (10% of T cells, 3% of B cells) expressed Azami green. In contrast, 54% of the myeloid cells and 76% of the hematopoietic progenitors present in the culture were positive for the fluorescent marker (data not shown), demonstrating that SeV preferentially transduces the less abundant hematopoietic progenitors and myeloid cells under these culture conditions.

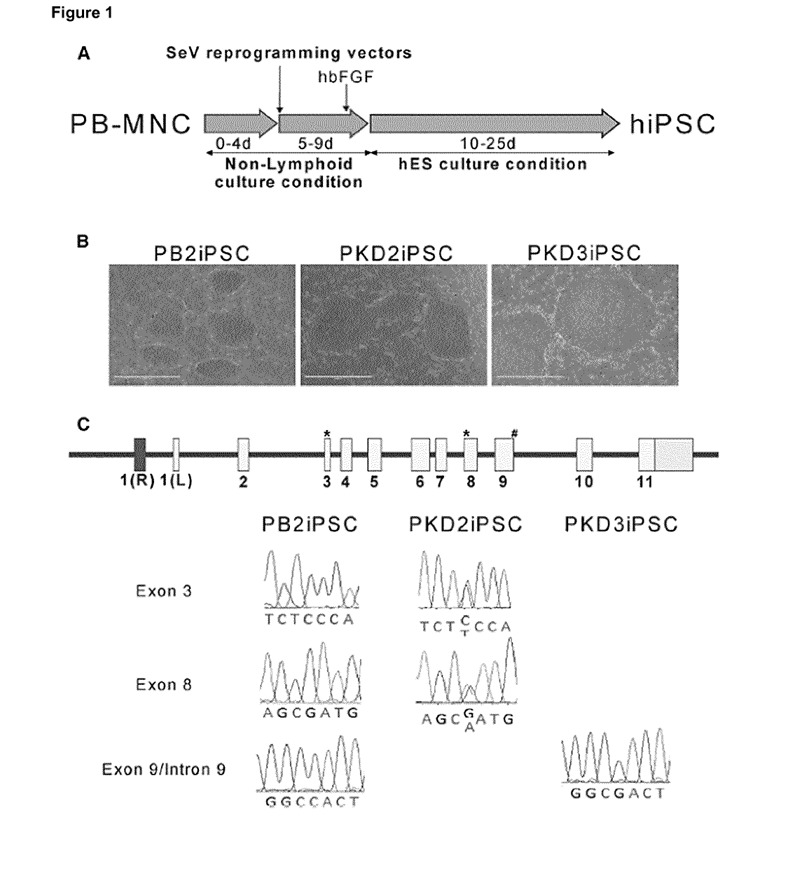

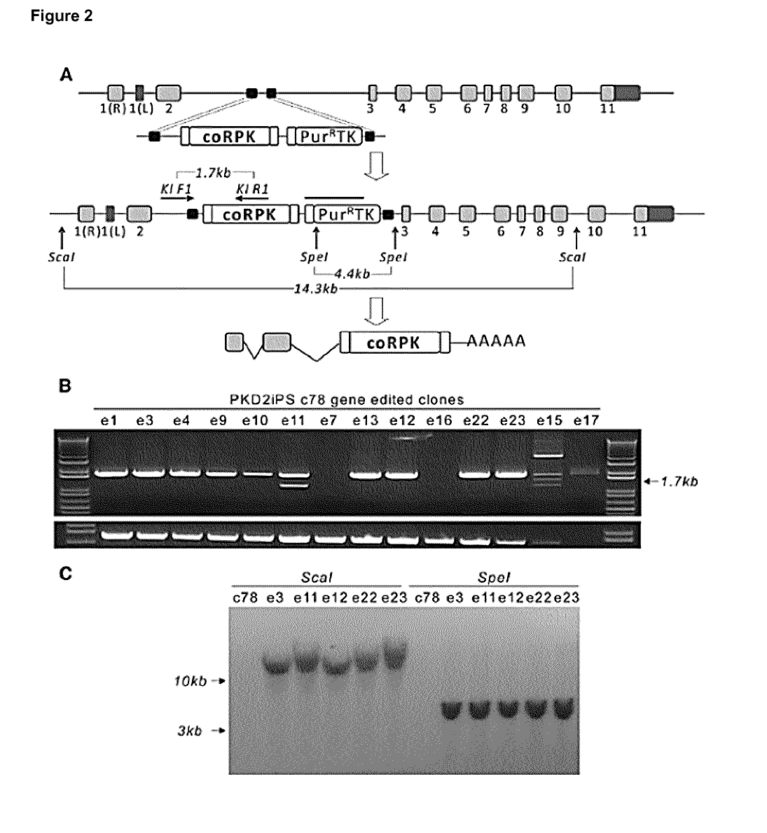

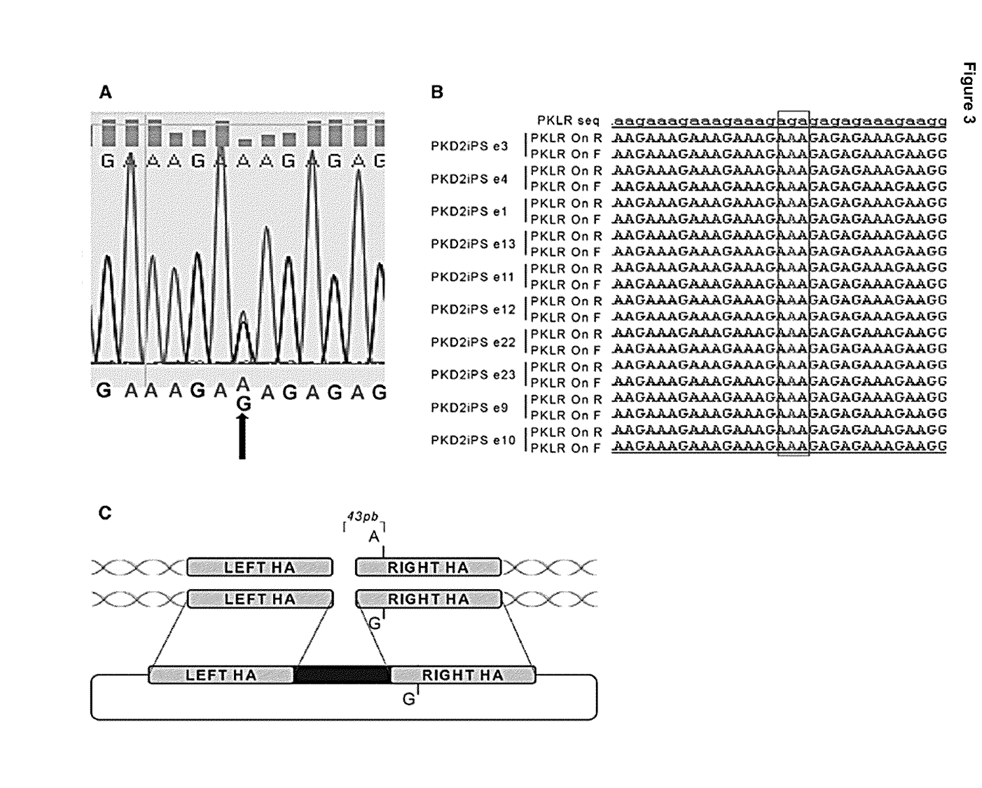

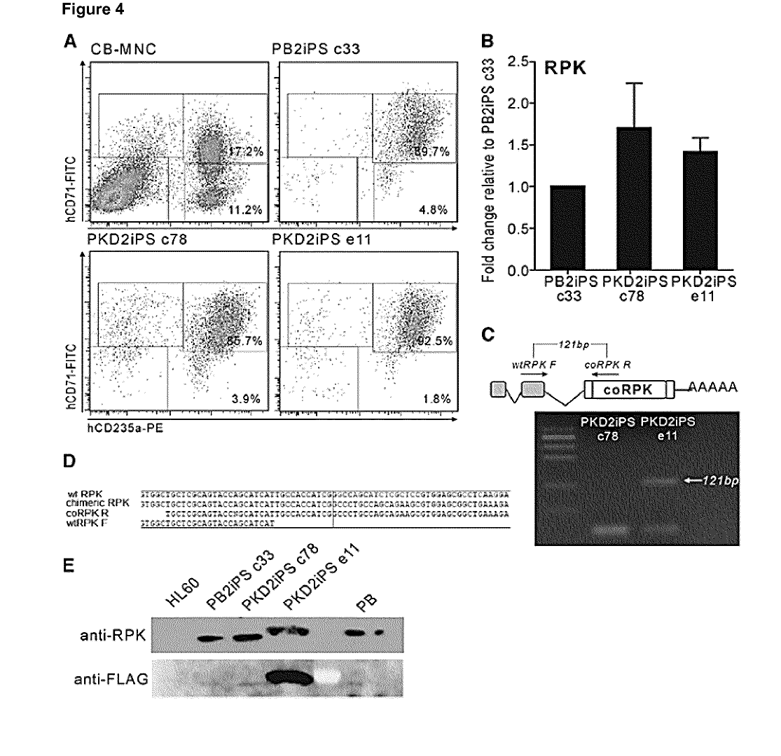

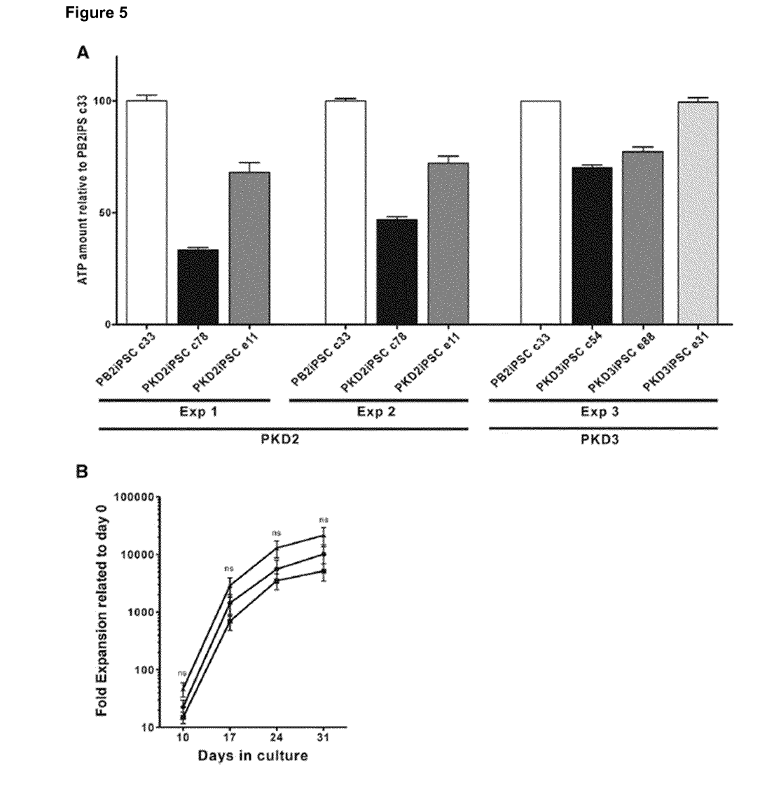

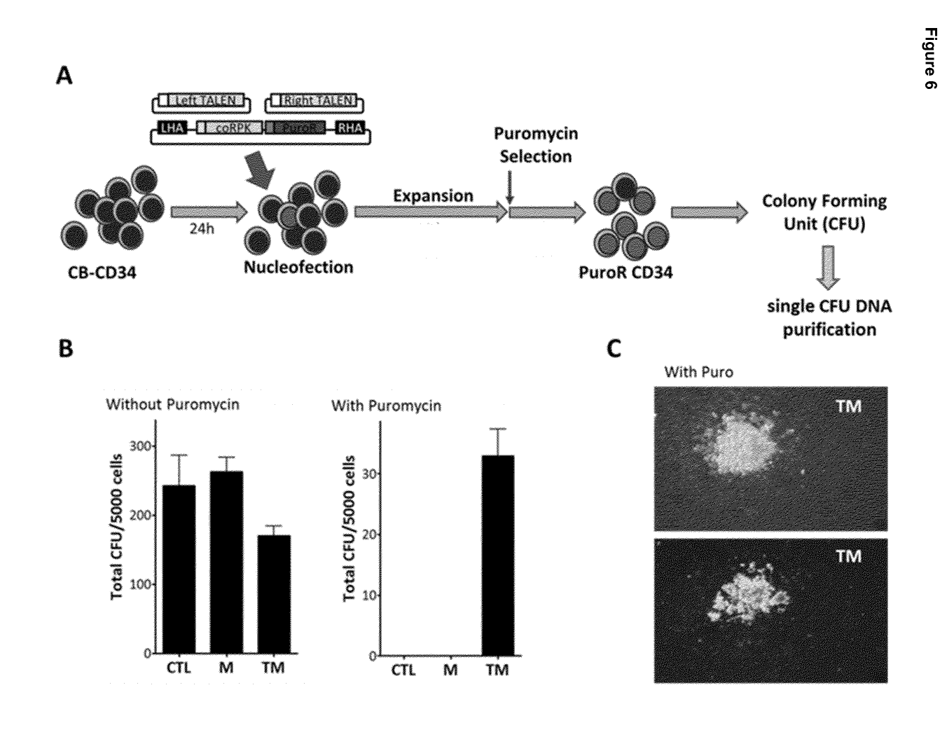

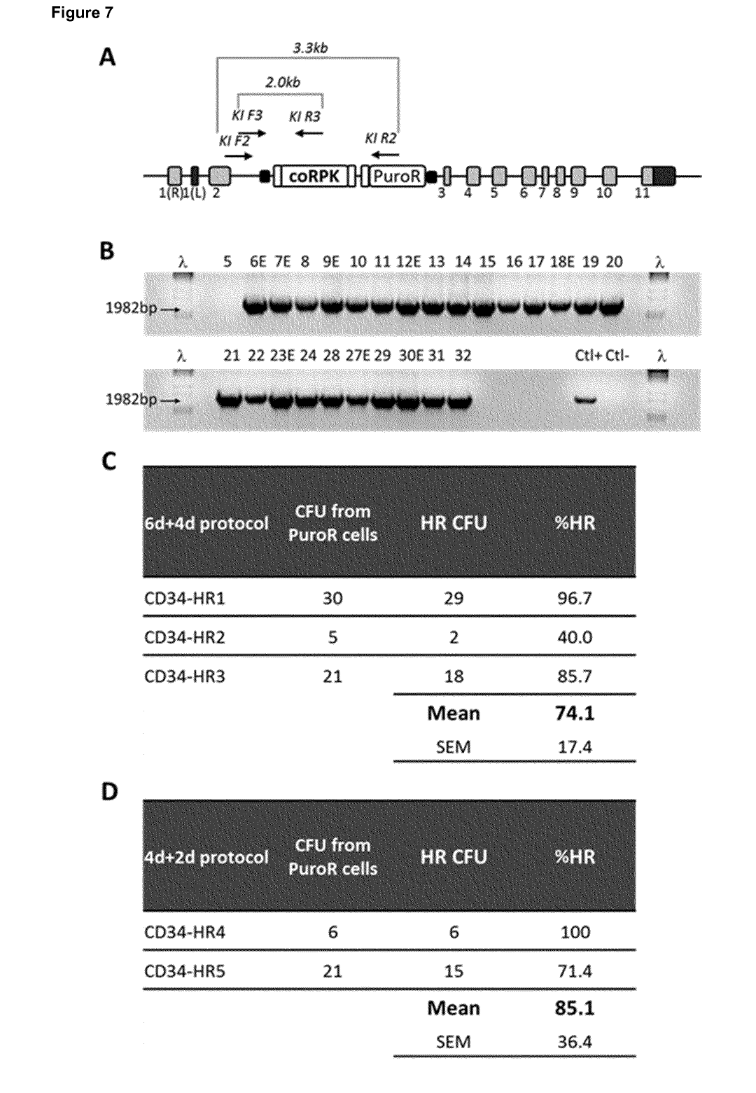

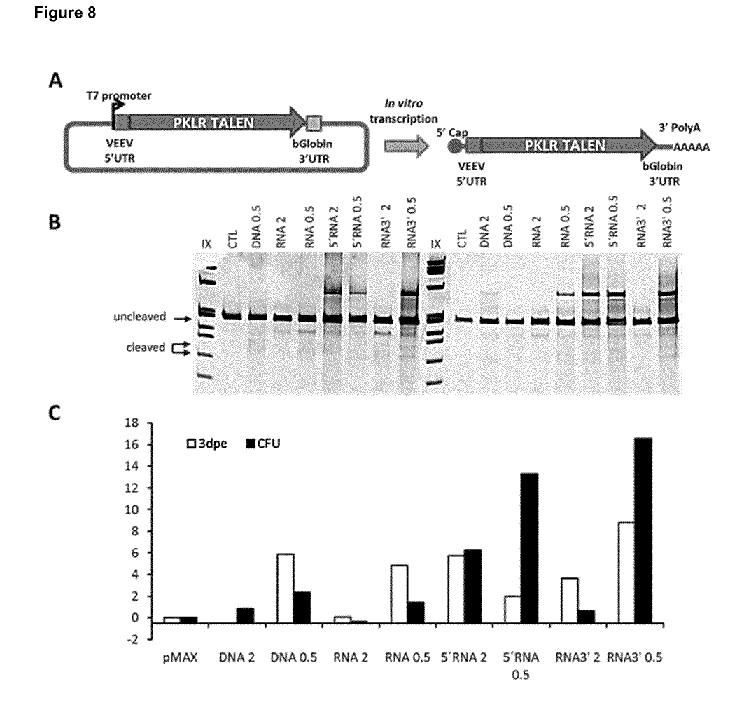

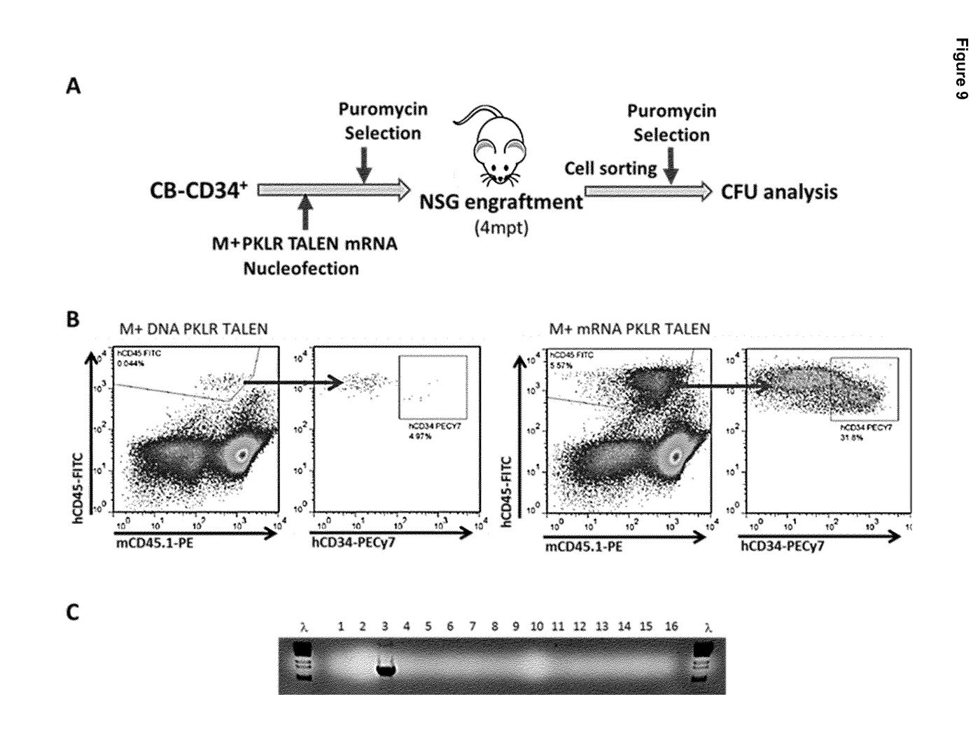

This transduction protocol was then used to reprogram PB-MNCs from healthy donors and PKD patients by SeV encoding the four “Yamanaka” reprograming factors (OCT3/4, KLF4, SOX2, and c-MYC; FIG. 1A). ESC-like colonies were obtained from one healthy donor (PB2) and from samples from two PKD patients (PKD2 and PKD3) PB-MNCs. Up to 20 ESC-like colonies derived from PB2, 100 from PKD2 and 50 from PKD3 were isolated and expanded (FIG. 1B). The complete reprogramming of the different established lines toward embryonic stem (ES)-like cells was evaluated. RT-PCR gene expression array verified a similar expression level of the main genes involved in pluripotency and self-renewal in our reprogramed cells and in the reference human ESC line H9. The ES markers OCT3/4, SSEA4, and Tra-1-60 were also corroborated by fluorescence-activated cell sorting (FACS) and immunofluorescence. Unmethylated status of NANOG and SOX2 promoters was confirmed by pyrosequencing.

NANOG promoter was strongly demethylated in lines derived from PB2, PKD2, and PKD3. Surprisingly, the SOX2 promoter was already unmethylated in PB-MNCs. Furthermore, the pluripotency of these lines derived from PB-MNCs was affirmed by their ability to generate teratomas into NOD.Cg-Prkdc^{scid}IL2rg^{tm/Wjl}/SzJ (NSG) mice, where all the mice injected developed teratomas showing tissues from the three different embryonic layers. These data confirmed the reprogrammed lines as bona fide iPSC lines denoted as PB2iPSC, PKD2iPSC, and PKD3iPSC. Additionally, the presence of the wild-type (WT) sequence or patient specific mutations in the different human iPSC lines generated was confirmed by Sanger sequencing of the corresponding genome loci (FIG. 10). PKD2iPSC showed the two heterozygous mutations in exon 3 (359C>T) and exon 8 (1168G >A), and PKD3iPSC carried the homozygous mutation in the splicing donor sequence of exon 9/intron 9 (IVS9(+1)G>C) characterized in the patients. These mutations could not be detected in peripheral-blood-derived induced pluripotent stem cells (PBiPSCs), which showed the expected WT sequences (FIG. 1C).

To confirm the absence of ectopic reprogramming gene expression, we analyzed the disappearance of SeV vectors in the generated iPSCs. The presence of the ectopic proteins could be tracked by the persistence of the fluorescent marker, as the SeV expressing Azami green was co-transduced together with the reprogramming vectors. Azami green expression was only detected in non-reprogramed, fibroblast-like cells in early passages. Green fluorescence disappeared in all the iPSC colonies. Importantly, SeV mRNA was not detected in iPSCs derived from PB-MNCs in late passages.

In addition, to check whether the established protocol did allow preferential reprogramming in myeloid and/or progenitor cells, Tcell receptor (TCR) and immunoglobulin heavy-chain genome rearrangements were studied on the iPSC generated. None of the analyzed iPSC clones (PB2iPSC c33, PKD2iPSC c78, PKD3iPSC c14, PKD3iPSC c10, and PKD3iPSC c35) had any T or B rearrangements, meaning that iPSC clones were generated from neither T nor B lymphocytes. These results guarantee the SeV-based reprograming system as the best option in reprogramming peripheral blood, as the reprograming vectors are cleared after iPSC generation, and the iPSC are generated from non-lymphoid cells. To continue with the following gene-editing steps clones from PB2, PKD2, and PKD3, we randomly selected PB-MNCs.

### Example 2

### TALEN-Based Gene Editing in the PKLR Locus of PKDiPSCs

To achieve correction of PKDiPSCs, we used a knock-in gene-editing strategy based on inserting a therapeutic matrix containing a partial codon-optimized (cDNA) RPK gene covering exons 3 to 11 (SEQ ID NO:5), fused to a FLAG tag (SEQ ID NO: 6) and preceded by a splice acceptor signal (SEQ ID NO:7). Additionally, a positive-negative selection cassette containing a puromycin (Puro) resistance/thymidine kinase (TK) fusion gene driven by mouse phosphoglycerate kinase (mPGK) promoter (SEQ ID NO:8) was included downstream of the partial (cDNA) RPK. These elements were flanked by two homology arms (SEQ ID NO:9 and 10) matching sequences in the second intron of the PKLR gene (FIG. 2A).

In order to increase the efficiency of gene editing, we developed a PKLR-specific TALEN targeting a specific genomic sequence in the second intron (SEQ ID NO:11) flanked by the homology arms. Nuclease activity of the PKLR TALEN in the target sequence was verified by surveyor assay after nucleofecting both subunits of the nuclease in PKD2iPSC and PKD3iPSC.

In two independent experiments, two iPSC lines from two different PKD patients, PKD2iPSC c78 and PKD3iPSC c54, were nucleofected with a control plasmid or with the developed matrix (from now on called therapeutic matrix or homologous recombination (HR) matrix) alone or together with two different doses of PKLR TALEN (1.5 or 5 mg of each PKLR TALEN subunit). Two days later, Puro was added to the media for 1 week. Puro-resistant (PuroR) colonies, with a satisfactory morphology appeared and were individually picked and subcloned. Most of the PuroR colonies were identified from cells nucleofected with both the matrix and the PKLR TALEN subunits, although some colonies grew out after receiving only the therapeutic matrix. There was no difference in the number of PuroR colonies between PKDiPSC lines from the different patients. To confirm target insertion of the therapeutic matrix in the second intron of the PKLR gene, we performed specific PCR analyses (FIG. 2A). The expected PCR product was detected in 10 out of 14 PuroR clones from PKD2iPSC c78 and 31 out of 40 PuroR clones from PKD3iPSC c54 (FIG. 2B). Taken together, we estimated an HR frequency among the PuroR clones of above 75% for the two reprogramed patients (Table 1).

TABLE 1
Efficacy of Homologous Recombination in PKD2iPSCs and PKD3iPSCs
and Indels Analysis in the Untargeted Allele

| | | | Percentage of | Percentage of |
|---|---|---|---|---|
| | | Percentage | Gene-Edited | Gene-Edited |
| | | of | Clones | Clones with |
| | Puro^{R} | Gene-Edited | Targeted | Indels in the |
| | Clones | Clones | Biallelically | Untargeted Allele |
| PKD2iPSCs | 13 | 77% | 0% | 40% |
| PKD3iPSCs | 40 | 75% | 11% | 31% |

In addition, two PuroR clones from PKD3iPSC c54 clone nucleofected with the therapeutic matrix alone were positive for knock-in, estimating an efficiency of 0.6 edited per 1×10⁵ nucleofected cells. Despite detecting HR without nucleases, the HR frequency was boosted almost five times (2.85 edited PKD3iPSC per 1×10⁵ nucleofected cells) when the PKLR TALEN was added. Additionally, knock-in insertion of the therapeutic matrix was verified by Southern blot (FIG. 2C), confirming a single insertion in the desired genomic locus.

Next, we tested whether the PKLR TALEN was also cutting the untargeted allele. Up to 40% of PKD2 and 31% of PKD3 edited clones carried insertions-deletions (indels) in the untargeted allele of the PKLR TALEN target site (Table 1), demonstrating the high efficacy of this PKLR TALEN. Moreover, 3 out of 40 edited clones from PKD3iPSC were targeted biallelically as determined when both the targeted allele and the untargeted were analyzed in a single PCR. In contrast, no edited PKD2iPSC clones showed biallelic targeting.

In order to check the specificity of the PKLR TALEN, we looked for potential off-target cutting sites in the different edited PKDiPSC clones. By in silico studies, we found five hypothetical off-target sites for this TALEN. These five off-targets can be recognized by the two subunits matched as homodimers or heterodimer, where the left subunit can join the right subunit or each subunit can join a different spacer sequence and length. All the potential off-targets had at least five mismatched bases, which makes the recognition by the TALEN unlikely. To confirm the specificity of the TALEN, we amplified genomic DNA from several edited PKD2iPSC and PKD3iPSC clones and Sanger sequenced around four offtargets (off-targets 1, 2, 4, and 5). None of the analyzed clones showed any indels in any of the off-targets analyzed. Off-target 3 could not be amplified by PCR. Nevertheless, as the first base in the 50 recognition sites of the off-target 3 was an A, the recognition of this offtarget by the PKLR TALEN is strongly reduced (Boch et al., 2009). This high specificity together with the high efficacy of PKLR TALEN confirms the feasibility of the developed TALEN and therapeutic matrix to promote HR in the PKLR locus.

Finally, we verified the pluripotency of the edited iPSCs after gene editing by in vivo teratoma formation into NSG mice. Edited clones were able to generate teratomas with tissues from the three embryonic layers. More importantly, human hematopoiesis, demonstrated by the presence of cells expressing the human CD45 panleukocytary marker (4.54% of the total teratoma forming cells) and human progenitors (CD45+CD34+; 2.74% of the total hCD45+ cells) derived from edited PKD3iPSC e31 teratomas could also be detected in vivo. Altogether, the data confirm the use of PKLR TALEN to edit the PKLR gene in PKDiPSCs without affecting their pluripotent properties.

### Example 3

### A Single-Nucleotide Polymorphism Leads to Allele-Specific Targeting

While evaluating the presence of indels in the untargeted allele by Sanger sequencing, we identified the existence of a g.[2268A >G] SNP 43 bases apart from the PKLR TALEN cutting site in PKD2iPSC (FIG. 3A). Interestingly, the untargeted allele from all the edited PKD2iPSC clones (ten out of ten) carried the previously mentioned SNP, suggesting an impediment of the allele carrying the SNP variant to carry out HR. Moreover, no biallelic targeting was detected in any PKD2iPSC edited clone. On the contrary, 3 out of 31 edited PKD3iPSC clones without any SNP in the homology genomic area were targeted in both alleles.

### Example 4

### Genetic Stability of PKDiPSCs and Gene-Edited PKDiPSCs

We wanted to study whether the whole process of reprogramming plus gene editing was inducing genetic instability in the resulting cells. As a first approach, we performed karyotyping of the different iPSC lines and confirmed normal karyotype in all cases. However, to have a clearer assessment, we monitored the genetic stability throughout all the process, including iPSC generation and gene-editing correction, by comparative genomic hybridization (CGH) and exome sequencing. PB-MNCs from a PKD2 patient, reprogrammed PKD2iPSC c58, and edited PKD2iPSC ell were selected as representatives of each step. Copy-number variations (CNVs) were defined in these samples after comparing with a reference genomic DNA. Among the total CNVs identified, 31 were present in the original PB-MNC from PKD2, 34 CNVs were detected in PKD2iPSC c78, and 32 in PKD2iPSC ell (Table 2). Twenty-three CNVs detected in PKD2iPSC c78 were already present in PKD2 PB-MNCs, indicating the mosaicism of the original patient sample. On the other hand, only four CNVs present in PKD2iPSC c78 and PKD2iPSC ell were not detected in the primary sample. Of note, these four CNV were at chromosomes 1q44, 2p21, 3p12.3-p12.1, and Xp11.22, involving genes such as ROBO1, GBE1, TCEA1, LYPLA1, DLG2, PLEKHA5, and AEBP2 (Table 2).

TABLE 2
Copy-Number Variations and Exome Variants Detected by CGH and Exome Sequencing in Edited PKD2iPSCs
CGH Analysis

| Number | Chromosome | Cytoband | Size (bp) | Type | Present in PKD2iPSC c78 | Present in PKD2 PB-MNCs |
|---|---|---|---|---|---|---|
| 1 | 1 | q44 | 60,641 | DEL | no | no |
| 2 | 3 | p12.2-p12.1 | 3,931,633 | LOH | yes | no |
| 3 | 8 | q11.23 | 169,460 | AMP | yes | no |
| 4 | 11 | q14.1 | 113,264 | DEL | yes | no |
| 5 | 12 | p12.3 | 1,182,747 | AMP | yes | no |
| 6 | 17 | q21.31 | 199,747 | AMP | yes | no |
| 7 | X | p11.22 | 6,030 | AMP | no | no |

Exome Sequencing

| Number | Chromosome | Reference Base | Altered Base | Gene | Type | Present in PKD2iPSC c78 |
|---|---|---|---|---|---|---|
| 1 | 9 | — | TGCCTCCACCACACC | PHF2 | nonframeshift insertion | no |
| 2 | 16 | G | T | ZNF747 | nonsynonymous SNV | no |
| 3 | 6 | G | C | SNX3 | nonsynonymous SNV | no |
| 4 | 22 | A | T | TUBGCP6 | nonsynonymaus SNV | no |
| 5 | 10 | A | G | TARC2 | nonsynonymous SNV | no |
| 6 | 7 | C | A | TNRC18 | stop-gain SNV | no |
| 7 | 18 | C | A | MBD2 | nonsynanymous SNV | yes |
| 8 | 18 | C | A | MBD2 | nonsynonymous SNV | yes |
| 9 | 9 | G | T | RUSC2 | nonsynonymous SNV | yes |
| 10 | 11 | G | A | APOA5 | nonsynonymaus SNV | yes |

SNV, single-nucleotide variation.
See also Tables S4 and S5.

More importantly, only two CNVs appeared after gene-editing that were not present in the original iPSC clone. The first one was a deletion of 6.6 kb that include several olfactory receptor genes (such asOR2T11, OR2T35, or OR2T27), and the second CNV was anamplification of 0.6 kb that includes the FGD1 gene. Additionally, sequences surrounding these two CNVs in PKD2iPSC e11 have more than eight mismatches with the PKLR TALEN recognition site, suggesting that these genomic alterations were not produced by gene editing.

Moreover, we analyzed the presence of CNVs in PKD3iPSC before and after gene editing to confirmthe potential harmless effect in the genomic stability of PKLR TALEN activity (Table S4). Edited clonePKD3iPSCe31 (biallelically targeted) showed 10 out 11 CNVs of the parental PKD3iPSC c54, and PKD3iPSC e88 (monoallelically targeted) showed two new CNVs. Furthermore, none of the CNVs present in the edited PKD2iPSC e11 were present in any of these two PKD3iPSC edited clones, which suggests that PKLR TALEN does not induce any specific CNVs in PKDiPSC clones.

Simultaneously, the three PKD2 samples were assayed using the Illumina HiSeq 2000 system for exome sequencing. After bioinformatics analysis by comparing the sequencing data with a human genome reference, PKD2 PB-MNCs showed 68,260 changes in their sequences, PKD2iPSC c78 68,542, and PKD2iPSC e11 67,728. Only ten of all variants detected in PKD2iPSC e11 were in exonic regions, included in the SNP database, and not identified in PKD2 PB-MNCs (Table 2). Additionally, four of them were also detected in PKD2iPSC c78. In order to verify the presence of these mutations by Sanger sequencing, we PCR amplified and sequenced these regions. Only the mutations in the RUSC2, TACR2, and in APOA5 genes could be confirmed by sequencing (data not shown). None of the ten variants were included in the COSMIC database (Wellcome Trust Sanger Institute, 2014), which includes all the known somatic mutations involved in cancer.

Overall, genetic stability analysis confirmed the safety o our gene editing approach. All the genetic alterations identified were present in the PB-MNCs or generated during their reprogramming or iPSC expansion. Moreover, none of the confirmed alterations could be associated with potentially dangerous mutations.

### Example 5

### Gene-Edited PKDiPSCs Recover RPK Functionality

Once the knock-in integration was confirmed, we assessed the PK phenotypic correction of the gene-edited iPSCs. We induced the erythroid differentiation of different iPSC lines from a healthy donor iPSC line (PB2iPSC c33), PKD iPSC lines derived from both patients (PKD2iPSC c78 and PKD3iPSC c54), and the corresponding edited clones (monoallelically edited PKD2iPSC ell and PKD3iPSC e88 and a biallelically targeted PKD3iPSC e31). Characteristic hematopoietic progenitor markers, such as CD43, CD34, and CD45, started to appear over time and were expressed in a similar proportion of cells. Erythroid cells were clearly observed in the cultures, and the specific erythroid combination of CD71 and CD235a antigens was expressed on the majority of cells after 21 days of differentiation (FIG. 4A). Moreover, cells derived from all iPSC lines analyzed at day 31 of differentiation, showed a similar globin pattern, in which a- and γ-globins were predominant with a small amount of β-globin, and residual embryonic ε- and z-globins detected, confirming the erythroid differentiation of these pluripotent lines. More importantly, the erythroid cells derived from the three iPSC lines were able to express RPK (FIGS. 4B and 4E). It is noteworthy that no alteration in the expression of proximal genes in the edited erythroid cells was confirmed by qRT-PCR.

The presence of chimeric transcripts in all of the edited PKDiPSC lines was confirmed by RT-PCR. Primers recognizing a sequence in the second endogenous exon of the PKLR gene and in the partial codon-optimized (cDNA) RPK were able to produce an amplicon with the correct size, specifically in erythroid cells derived from gene-edited PKDiPSCs (FIG. 4C). This amplicon was sequenced and the joint between both parts of the mRNA, coming from the transcription of the endogenous and the exogenous sequences, was detected (FIG. 4D). Additionally, the presence of RPK was demonstrated by western blot in the erythroid cells derived from all of the edited iPSC lines derived from PKD2iPSC c78 (PKD2iPSC e11; FIG. 4E) and from PKD3iPSC c54 (PKD3iPSC e88 and PKD3iPSC e31). Interestingly, although (mRNA) RPK could be detected in erythroid cells derived from all the iPSC lines derived from PKD3, RPK protein was not detected in PKD3iPSC c54, probably due to the severity of the mutation in terms of RNA translation. However, the gene editing of PKD3iPSC restored RPK protein expression either in the bialellic (PKD3iPSC e31) and monoallelic (PKD3iPSC e88) edited lines. Moreover, both the level of the chimeric transcript and the RPK protein were higher in the biallelically targeted clone PKD3iPSC e31 than in the monoallelic PKD3iPSC e88. It is worth it mentioning that flagged RPK was detected in erythroid cells generated after gene editing of PKDiPSCs (FIG. 4E), confirming the origin of the RPK protein from the edited genome.

Finally, the recovery in metabolic function of the corrected cells was assessed in the differentiated cells by conventional biochemical analysis as well as by liquid chromatography mass spectrometry (LC-MS) (FIG. 5). The ATP level in erythroid cells derived from the monoallelically edited PKDiPSCs (PKD2iPSC e11 and PKD3iPSC e88) was augmented after gene editing (FIG. 5A), reaching an intermediate level between that observed in erythroid cells from WT iPSCs and their respective patient-specific iPSC lines. Additionally, erythroid cells derived from the biallelically targeted PKD3iPSC e31 restored the ATP level completely up to healthy values (FIG. 5A). In edited erythroid cells, other glycolytic metabolites, such as 2,3-diphosphoglyceric acid, 2-phosphoglyceric acid, pyruvic acid, and L-lactic acid, reached levels between those of control and deficient erythroid cells derived from PB2iPSCs and PKDiPSCs. In addition, we obtained up to 2-3 10⁴-fold expansion of cells in 1 month, meaning that up to 20,000 erythroid cells could be generated from a single iPSC (FIG. 5B). As expected, no statistical differences were observed between the different iPSCs, indicating that RPK deficiency only affects the last steps of the erythroid differentiation, where no proliferation is taking place. Altogether, our data validate the effectiveness of this knock-in approach to express a corrected RPK protein and demonstrate its potential to therapeutically correct the PKD phenotype and generate large numbers)(10⁹-10¹⁰) of differentiating cells required for comprehensive biochemical and metabolic analyses during their maturation, or even for a potential therapeutic use.

### Example 6

### Peripheral Blood Samples and Reprogramming

Peripheral blood from PKD patients and healthy donors was collected in routine blood sampling from Hospital Clinico Infantil Universitario Niño Jesús (Madrid, Spain), Centro Hospitalario de Coimbra (Coimbra, Portugal), and the Medical Care Service of CIEMAT (Madrid, Spain). All samples were collected under written consent and institutional review board agreement. PB-MNCs were isolated by density gradient using Ficoll-Paque (GE Healthcare). PB-MNCs were pre-stimulated for 4 days in StemSpan (STEMCELL Technologies) plus 100 ng/ml human stem cell factor (SCF), 100 ng/ml hFLT3L, 20 ng/ml hTPO, 10 ng/ml G-CSF, and 2 ng/ml human IL-3 (Peprotech) (FIG. 1A). Cells were then transduced with a mix of SeV, kindly provided by DNAvec (Japan), expressing OCT3/4, KLF4, SOX2, c-MYC, and Azami Green, each at a MOI of 3. Transduced cells were maintained for four more days in the same culture medium and then supplemented with 10 ng/ml basic fibroblast growth factor (FGF). Five days after transduction, cells were collected and seeded on irradiated human foreskin fibroblast (HFF-1)-coated (ATCC) culture plates with human ES media (knockout DMEM, 20% knockout serum replacement, 1 mM L-glutamine, and 1% nonessential amino acids [all from Life Technologies]), 0.1 mM b-mercaptoethanol (Sigma-Aldrich), and 10 ng/ml basic human FGF (Peprotech). Human ES media was changed every other day. When human ES-like colonies appeared, they were selected under the stereoscope (Olympus) and a clonal culture from each colony was established.

### Example 7

### Gene Editing in iPSCs

iPSCs were treated with Rock inhibitor Y-27632 (Sigma) before a single-cell suspension of iPSCs was generated by StemPro Accutase (Life Technologies) treatment and then nucleofected with 1.5 mg or 5 mg of each PKLR TALEN subunit with or without 4 mg HR matrix by Amaxa Nucleofector (Lonza) using the A23 program. After nucleofection, cells were seeded into a feeder of irradiated PuroR mouse embryonic fibroblasts in the presence of Y-27632, and 48 hr after transfection, puromycin (0.5 mg/ml) was added to human ES media. Newly formed PuroR-PKDiPSC colonies were picked individually during a puromycin selection period of 6-10 days. PuroR-PKDiPSC colonies were expanded and analyzed by PCR and Southern blot to detect HR (FIGS. 2B and 2C).

### Example 8

### Erythroid Differentiation

Erythroid differentiation from iPSC lines was performed using a patented method (WO/2014/013255). In brief, we used a multistep, feeder-free protocol developed by E.O. Before differentiation, normal, diseased, and corrected iPSCs were maintained in StemPro medium (Life Technologies) with the addition of 20 ng/ml basic FGF on a matrix of recombinant vitronectin fragments (Life Technologies) using manual passage. For initiation of differentiation, embryoid bodies (EBs) were formed in Stemline II medium (Sigma Aldrich) with BMP4, vascular endothelial growth factor (VEGF), Wnt3a, and activin A. In a second step, hematopoietic differentiation was induced by adding FGFa, SCF, IGF2, TPO, and heparin to the EB factors. After 10 days, hematopoietic progenitors were harvested and replated into fresh Stemline II medium supplemented with BMP4, SCF, Flt3 ligand, IL-3, IL-11, and erythropoietin (EPO) to direct differentiation along the erythroid lineage and to support extensive proliferation. After 17 days, cells were transferred into Stemline II medium containing a more specific erythroid cocktail that included insulin, transferrin, SCF, IGF1, IL-3, IL-11, and EPO for 7 days. In a final maturation step of 7 days (days 24-31), cells were transferred into IMDM with insulin, transferrin, and BSA and supplemented with EPO. Cells were harvested for analysis on days 10, 17, 24, and 31.

### Example 9

### Gene Editing of Human Hematopoietic Progenitors in the PKLR Locus

In order to research the feasibility of applying our knock-in gene editing approach in human hematopoietic progenitors, the iPSC gene editing protocol was adapted to be performed with hematopoietic progenitors.

Material and methods: Cord Blood CD34⁺ (CB-CD34) cells were cultured in StemSpan (StemCell Technologies)/0.5% Penicillin-Streptomycin (Thermo Fisher Scientific)/100 ng/ml SCF/100 ng/ml FLT3L/100 ng/ml TPO (all cytokines from Peprotech) for 24 hours before being nucleofected by the matrix and PKLR TALEN. 1'10⁶ CB-CD34 were nucleofected with 5 μg homologous recombination matrix (M) or/and 2.5 μg of each PKLR TALEN subunit (T) targeting a specific sequence in the second intron of the PKLR gene by Amaxa™ Nucleofector™ II (Lonza) using U08 program. Then, the CB-CD34 cells were expanded for 6 days and selected with puromycin (Sigma-Aldrich) for another additional 4 days. Semisolid cultures for the identification of hematopoietic progenitors (colony forming unit [CFU] assay) using HSC-CFU media (Myltenyi) was performed and the colonies were counted and picked for their analysis for specific integration by Nested-PCR. A schematic representation of the gene editing protocol is provided in FIG. 6A.

Results: There was a high mortality, pointed out by a reduction in the total number of cells and in the total number of CFUs, when CB-CD34 were electroporated by the matrix and the PKLR TALEN compared with sham electroporated (CTL) or electroporated only with the PKLR TALEN. This mortality was due to the toxicity associated to the DNA electroporation (FIG. 6B). However, CFUs derived from Puro^{R} progenitors were identified only when CB-CD34 cells were electroporated with the matrix plus PKLR TALEN. Interestingly, Puro^{R} progenitors gave rise either myeloid or erythroid CFUs (FIG. 6C).

### Example 10

### Specific Integration of the Matrix in the PKLR Locus by Nested PCR

The specific integration of the matrix in the PKLR locus was determined by nested PCR. Material and methods: Individual CFUs were picked and analyzed to identify the specific integration of the matrix in the PKLR locus by nested PCR (FIG. 7A). Nested PCR was used to increase sensitivity and reduce non-specific amplification. The Nested PCR designed to analyze gene editing in the PKLR locus. The nested PCR involved two sets of primers:

first set, KI F2
(SEQ ID NO: 12: ACTGGGTGATTCTGGGTCTG)
and
KI R2
(SEQ ID NO: 13 GGGGAACTTCCTGACTAGGG);
and
second set, KI F3
(SEQ ID NO: 14: GCTGCTGGGGACTAGACATC)
and
KI R3
(SEQ ID NO: 15: CGCCAAATCTCAGGTCTCTC).

These were used in two successive runs of PCR. The second set of primers amplified a secondary target of 2.0kb within the first run product of 3.3kb. The two forward primers recognized genome endogenous PKLR sequence downstream from matrix integration site and the reverse primers bound Puro^{R} cassette and coRPK cassette respectively in the integrated matrix. Nested PCR was performed using Herculase II Fusion DNA Polymerase (Agilent). In order to improve the gene editing strategy, the knock-in protocol was shortened in order to maintain the hematopoietic stem cell potential. Expansion period was shortened from 6 to 4 days and the selection period from 4 to 2 days (4d+2d protocol), FIG. 7D.

Results: Most CFUs derived from Puro^{R} human hematopoietic progenitors were correctly gene edited with our strategy (FIG. 7A). FIG. 7B shows the amplified sequence of 2.0kb resulting from the Nested PCR analysis of CFUs derived from CB-CD34 electroporated with TM and selected with puromycin. Up to 74% of the analyzed CFUs were positive for the knock-in integration (6d+4d protocol), FIG. 7C. In order to improve the gene editing strategy, the knock-in protocol was shortened in order to maintain the hematopoietic stem cell potential. When expansion period was shortened from 6 to 4 days and the selection period from 4 to 2 days (4d+2d protocol), the percentage of gene edited human hematopoietic progenitors did not change however significantly (up to 71% CFUs were positive for the specific integration, FIG. 7D). Moreover, some primitive CFUs (GEMM-CFU) could be identified, whereby primitive human hematopoietic progenitors were gene edited with our protocol.

### Example 11

### Improvement of Delivery of PKLR TALEN

To reduce the toxicity associated to nucleofected DNA, the use of PKLR TALEN as mRNA has been studied. To improve the stability of the PKLR TALEN mRNAs several modifications were introduced to either stabilize the mRNA (SEQ ID NO: 4, 3′UTR β-Globin) or to reduce the immune response against exogenous mRNAs (SEQ ID NO:3, 5′UTR VEEV, see Hyde et al, Science 14 Feb. 2014: 783-787).

Material and methods: CB-CD34 cells were nucleofected with either PKLR TALEN as plasmid DNA or as mRNA with different modifications (unmodified mRNA, 5′UTR VEEV mRNA and mRNA 3″UTR b-Globin) (FIG. 8A). 1×10⁵ CB-CD34 were nucleofected with either PKLR TALEN as plasmid DNA or as mRNA with different modifications (unmodified mRNA, 5′UTR VEEV mRNA and mRNA 3″UTR b-Globin), in vitro transcribed by mMESSAGE mMACHINE® T7 Ultra Kit (Thermo Fisher Scientific), using different amounts (0.5 μg or 2 μg) in a 4D-Nucleofector™ (Lonza). Surveyor assay (IDT) was performed three days after electroporation (FIG. 8B, left panel) or in CFUs derived from nucleofected hematopoietic progenitors (FIG. 8B, right panel). Surveyor® Mutation Detection Kits provide a simple and robust method to detect mutations and polymorphisms in DNA. The key component of the kits is Surveyor Nuclease, a member of the CEL family of mismatch-specific nucleases derived from celery. Surveyor Nuclease recognizes and cleaves mismatches due to the presence of single nucleotide polymorphisms (SNPs) or small insertions or deletions. The indels (insertions/deletions) obtained in the surveyor assay showed in FIG. 8B were evaluated by band densitometry and ratio of band intensities between cleaved and uncleaved bands (%), FIG. 8C.

Results: Interestingly, the highest targeting in PKLR locus was obtained when PKLR TALEN mRNA was modified by either 5′UTR VEEV or 3″UTR β-Globin. So, PKLR TALEN mRNA with 5′ and/or 3′ modifications was used in the subsequent experiments.

### Example 12

### Engraftment of Gene-Edited Human Hematopoietic Stem Cells in NSG Mice

The engraftment of gene-edited HSCs was assessed in NSG mice bone marrow four months after transplantation by determining by FACS the presence of human hematopoieitc cells (hCD45⁺) and human hematopoietic progenitors (CD45⁺/CD34⁺).

Material and methods: Fresh CB-CD34 cells were nucleofected by the HR matrix (M) plus either PKLR TALEN, as plasmid DNA or mRNAs carrying both mRNA modifications previously described. Puro^{R} cells expanded and drug selected as described above (4d+2d protocol) were transplanted intravenously into sub-lethally irradiated immunodeficient NSG mice (NOD.Cg-Prkdc^{scid} IIrg^{tm1Wjl}) (FIG. 4A). These animals allow the xenogenic engraftment of human hematopoietic stem cells and the generation of human mature hematopoietic cells. Four months after transplantation, human engraftment was analyzed by FACS by identificating human hematopoietic cells (hCD45⁺) over mouse hematopoietic cells (mCD45⁺) and human hematopoietic progenitors (CD45⁺/CD34⁺). CD45⁺/CD34⁺ cells were then isolated from the mouse bone marrow by cell sorting. Isolated human progenitors were cultured and puromycin selected and CFU assay was performed. Gene editing in these engrafted human hematopoietic progenitors was analyzed in individual CFUs by Nested PCR as described above.

Results: Human hematopoietic cells were identified in animals transplanted in CB-CD34 nucleofected with both matrix and PKLR TALEN as DNA (FIG. 9B, left panels), but this human engraftment (% hCD45⁺ cells) was below 0.5% of the total mouse bone marrow, with a small presence of human hematopoietic progenitors (% hCD45⁺hCD34⁺ cells). However, in the animals transplanted by PKLR TALEN as mRNA plus matrix (FIG. 9B, right panels), the human hematopoietic engraftment rose at 5.57% of the total mouse bone marrow cells. Moreover, a significant presence of human hematopoietic progenitors was observed. All together these data suggest a more favorable condition for human hematopoietic stem cell maintenance when nucleofection of mRNAs for the PKLR TALEN is used. To increase the resolution of the assay, a second round of puromycin selection was performed after isolating the population of human progenitors (hCD45⁺CD34⁺) from the mouse bone marrow. CFU assay was performed and these hematopoietic colonies were interrogated for knock-in integration on the expected genome site as previously described. One out 27 CFUs derived from engrafted human CD34 was positive for HR when the gene editing was mediated by PKLR TALEN mRNA (FIG. 9C). This indicates that PKLR gene editing was performed in human Hematopoietic Stem Cells, which kept their engraftment ability. Altogether point out the feasibility of our knock-in strategy through gene editing of Hematopoietic Stem Cells to correct PKD.

### BIBLIOGRAPHY

Aasen, T., Raya, A., Barrero, M. J., Garreta, E., Consiglio, A., Gonzalez, F., Vassena, R., Bilic, J., Pekarik, V., Tiscornia, G., et al. (2008). Efficient and rapid generation of induced pluripotent stem cells from human keratinocytes. Nat. Biotechnol. 26, 1276-1284.

Abyzov, A., Mariani, J., Palejev, D., Zhang, Y., Haney, M. S., Tomasini, L., Ferrandino, A. F., Rosenberg Belmaker, L. A., Szekely, A., Wilson, M., et al. (2012). Somatic copy number mosaicism in human skin revealed by induced pluripotent stem cells. Nature 492, 438-442.

Amabile, G., Weiner, R. S., Nombela-Arrieta, C., D'Alise, A. M., Di Ruscio, A., Ebralidze, A. K., Kraytsberg, Y., Ye, M., Kocher, O., Neuberg, D. S., et al. (2013). In vivo generation of transplantable human hematopoietic cells from induced pluripotent stem cells. Blood 121, 1255-1264.

Asp, J., Persson, F., Kost-Alimova, M., and Stenman, G. (2006). CHCHD7-PLAG1 and TCEA1-PLAG1 gene fusions resulting from cryptic, intrachromosomal 8q rearrangements in pleomorphic salivary gland adenomas. Genes Chromosomes Cancer 45, 820-828.

Ban, H., Nishishita, N., Fusaki, N., Tabata, T., Saeki, K., Shikamura, M., Takada, N., Inoue, M., Hasegawa, M., Kawamata, S., and Nishikawa, S. (2011). Efficient generation of transgene-free human induced pluripotent stem cells (iPSCs) by temperature-sensitive Sendai virus vectors. Proc. Natl. Acad. Sci. USA 108,14234-14239.

Beutler, E., and Gelbart, T. (2000). Estimating the prevalence of pyruvate kinase deficiency from the gene frequency in the general white population. Blood 95,3585-3588.

Boch, J., Scholze, H., Schornack, S., Landgraf, A., Hahn, S., Kay, S., Lahaye, T., Nickstadt, A., and Bonas, U. (2009). Breaking the code of DNA binding specificity of TAL-type III effectors. Science 326, 1509-1512.

Carroll, D. (2011). Genome engineering with zinc-finger nucleases. Genetics 188, 773-782.

Cavazza, A., Moiani, A., and Mavilio, F. (2013). Mechanisms of retroviral integration and mutagenesis. Hum. Gene Ther. 24, 119-131.

WellcomeTrust Sanger Institute (2014).COSMIC: catalog of somatic mutations in cancer. http://cancer.sanger.ac.uk/cancergenome/projects/cosmic/.

De Gobbi, M., Viprakasit, V., Hughes, J. R., Fisher, C., Buckle, V. J., Ayyub, H., Gibbons, R. J., Vernimmen, D., Yoshinaga, Y., de Jong, P., et al. (2006). A regulatory SNP causes a human genetic disease by creating a new transcriptional promoter. Science 312, 1215-1217.

Deyle, D. R., Li, L. B., Ren, G., and Russell, D. W. (2014). The effects of polymorphisms on human gene targeting. Nucleic Acids Res. 42, 3119-3124.

Fermo, E., Bianchi, P., Chiarelli, L. R., Cotton, F., Vercellati, C., Writzl, K., Baker, K., Hann, I., Rodwell, R., Valentini, G., and Zanella, A. (2005). Red cell pyruvate kinase deficiency: 17 new mutations of the PK-LR gene. Br. J. Haematol. 129,839-846.

Fusaki, N., Ban, H., Nishiyama, A., Saeki, K., and Hasegawa, M. (2009). Efficient induction of transgene-free human pluripotent stem cells using a vector based on Sendai virus, an RNA virus that does not integrate into the host genome. Proc. Jpn. Acad., Ser. B, Phys. Biol. Sci. 85, 348-362.

Garate, Z., Davis, B. R., Quintana-Bustamante, O., and Segovia, J. C. (2013). New frontier in regenerative medicine: site-specific gene correction in patient-specific induced pluripotent stem cells. Hum. Gene Ther. 24, 571-583.

Genovese, P., Schiroli, G., Escobar, G., Di Tomaso, T., Firrito, C., Calabria, A., Moi, D., Mazzieri, R., Bonini, C., Holmes, M. C., et al. (2014). Targeted genome editing in human repopulating haematopoietic stem cells. Nature 510, 235-240.

Gore, A., Li, Z., Fung, H. L., Young, J. E., Agarwal, S., Antosiewicz-Bourget, J., Canto, I., Giorgetti, A., Israel, M. A., Kiskinis, E., et al. (2011). Somatic coding mutations in human induced pluripotent stem cells. Nature 471,63-67.

Hussein, S. M., Batada, N. N., Vuoristo, S., Ching, R. W., Autio, R., Na\ddot{}rva\ddot{}, E., Ng, S., Sourour, M., Ha\ddot{}ma\ddot{}la\ddot{}inen, R., Olsson, C., et al. (2011). Copy number variation and selection during reprogramming to pluripotency. Nature 471, 58-62.

Karakikes, I., Stillitano, F., Nonnenmacher, M., Tzimas, C., Sanoudou, D., Termglinchan, V., Kong, C. W., Rushing, S., Hansen, J., Ceholski, D., et al. (2015). Correction of human phospholamban R14del mutation associated with cardiomyopathy using targeted nucleases and combination therapy. Nat. Commun. 6, 6955.

Li J, Song W, Pan G, Zhou J. “Advances in Understanding the Cell Types and Approaches Used for Generating Induced Pluripotent Stem Cells.” Journal of Hematology & Oncology 7 (2014): 50. PMC. Web. 7 Nov. 2016.

Loh, Y. H., Agarwal, S., Park, I. H., Urbach, A., Huo, H., Heffner, G. C., Kim, K., Miller, J. D., Ng, K., and Daley, G. Q. (2009). Generation of induced pluripotent stem cells from human blood. Blood 113, 5476-5479.

Lopez-Manzaneda S., Fañanas S., Nieto-Romero V., Roman-Rodriguez F., Fernandez-Garcia M., Pino-Barrio M. J., Rodriguez-Fornes F., Diez-Cabezas B., Garcia-Bravo M., Navarro S., Quintana-Bustamante O.r and Segovia J. C; TITLE: Gene Editing in Adult Hematopoietic Stem Cells; JOURNAL/BOOK TITLE: In “Modern Tools for Genetic Engineering” Editor MSD Kormann. INTECH publishing 2016. ISBN: 978-953-51-4654-4.

Meza, N. W., Alonso-Ferrero, M. E., Navarro, S., Quintana-Bustamante, O., Valeri, A., Garcia-Gomez, M., Bueren, J. A., Bautista, J. M., and Segovia, J. C. (2009). Rescue of pyruvate kinase deficiency in mice by gene therapy using the human isoenzyme. Mol. Ther. 17,2000-2009.

Nishimura, K., Sano, M., Ohtaka, M., Furuta, B., Umemura, Y., Nakajima, Y., Ikehara, Y., Kobayashi, T., Segawa, H., Takayasu, S., et al. (2011). Development of defective and persistent Sendai virus vector: a unique gene delivery/expression system ideal for cell reprogramming. J. Biol. Chem. 286,4760-4771.

Nishishita, N., Takenaka, C., Fusaki, N., and Kawamata, S. (2011). Generation of human induced pluripotent stem cells from cord blood cells. J. Stem Cells 6, 101-108. Park, I. H., Zhao, R., West, J. A., Yabuuchi, A., Huo, H., Ince, T. A., Lerou, P. H., Lensch, M. W., and Daley, G. Q. (2008). Reprogramming of human somatic cells to pluripotency with defined factors. Nature 451, 141-146.

Porteus, M. H., and Carroll, D. (2005). Gene targeting using zinc finger nucleases. Nat. Biotechnol. 23, 967-973.

Renkawitz, J., Lademann, C. A., and Jentsch, S. (2014). Mechanisms and principles of homology search during recombination. Nat. Rev. Mol. Cell Biol. 15, 369-383.

Rio, P., Ban~os, R., Lombardo, A., Quintana-Bustamante, O., Alvarez, L., Garate, Z., Genovese, P., Almarza, E., Valeri, A., Di´ez, B., et al. (2014). Targeted gene therapy and cell reprogramming in Fanconi anemia. EMBO Mol. Med. 6,835-848.

Sebastiano, V., Maeder, M. L., Angstman, J. F., Haddad, B., Khayter, C., Yeo, D. T., Goodwin, M. J., Hawkins, J. S., Ramirez, C. L., Batista, L. F., et al. (2011). In situ genetic correction of the sickle cell anemia mutation in human induced pluripotent stem cells using engineered zinc finger nucleases. Stem Cells 29, 1717-1726.

Seki, T., Yuasa, S., Oda, M., Egashira, T., Yae, K., Kusumoto, D., Nakata, H., Tohyama, S., Hashimoto, H., Kodaira, M., et al. (2010). Generation of induced pluripotent stem cells from human terminally differentiated circulating T cells. Cell Stem Cell 7, 11-14.

Song, B., Fan, Y., He, W., Zhu, D., Niu, X., Wang, D., Ou, Z., Luo, M., and Sun, X. (2015). Improved hematopoietic differentiation efficiency of gene-corrected beta-thalassemia induced pluripotent stem cells by CRISPR/Cas9 system. Stem Cells Dev. 24,1053-1065. Staerk, J., Dawlaty, M. M., Gao, Q., Maetzel, D., Hanna, J., Sommer, C. A., Mostoslaysky, G., and Jaenisch, R. (2010). Reprogramming of human peripheral blood cells to induced pluripotent stem cells. Cell Stem Cell 7, 20-24.

Suvatte, V., Tanphaichitr, V. S., Visuthisakchai, S., Mahasandana, C., Veerakul, G., Chongkolwatana, V., Chandanayingyong, D., and Issaragrisil, S. (1998). Bone marrow, peripheral blood and cord blood stem cell transplantation in children: ten years' experience at Siriraj Hospital. Int. J. Hematol. 68, 411-419.

Takahashi, K., Tanabe, K., Ohnuki, M., Narita, M., Ichisaka, T., Tomoda, K., and Yamanaka, S. (2007). Induction of pluripotent stem cells from adult human fibroblasts by defined factors. Cell 131, 861-872.

Tanphaichitr, V. S., Suvatte, V., Issaragrisil, S., Mahasandana, C., Veerakul, G., Chongkolwatana, V., Waiyawuth, W., and Ideguchi, H. (2000). Successful bone marrow transplantation in a child with red blood cell pyruvate kinase deficiency. Bone Marrow Transplant. 26, 689-690.

Ye, Z., Zhan, H., Mali, P., Dowey, S., Williams, D. M., Jang, Y. Y., Dang, C. V., Spivak, J. L., Moliterno, A. R., and Cheng, L. (2009). Human-induced pluripotent stem cells from blood cells of healthy donors and patients with acquired blood disorders. Blood 114, 5473-5480.

Yu, J., Vodyanik, M. A., Smuga-Otto, K., Antosiewicz-Bourget, J., Frane, J. L., Tian, S., Nie, J., Jonsdottir, G. A., Ruotti, V., Stewart, R., et al. (2007). Induced pluripotent stem cell lines derived from human somatic cells. Science 318, 1917-1920.

Zanella, A., Fermo, E., Bianchi, P., and Valentini, G. (2005). Red cell pyruvate kinase deficiency: molecular and clinical aspects. Br. J. Haematol. 130, 11-25.

Zanella, A., Bianchi, P., and Fermo, E. (2007). Pyruvate kinase deficiency. Haematologica 92, 721-723.

Zou, J., Mali, P., Huang, X., Dowey, S. N., and Cheng, L. (2011). Sitespecific gene correction of a point mutation in human iPS cells derived from an adult patient with sickle cell disease. Blood 118,4599-4608.

1. Cells isolated from a subject suffering from a metabolic disease affecting the erythroid lineage, wherein the mutation or mutations in the gene causing the metabolic disease present in said cells are corrected by gene-editing via a knock-in strategy where a partial cDNA is inserted in a target locus of said gene to express a chimeric mRNA formed by endogenous first exons and partial cDNA under the endogenous promoter control, and wherein said cells have the ability to differentiate into the erythroid lineage.

2. The cells of claim 1, wherein said cells are i) hematopoietic stem or progenitor cells, or ii) induced pluripotent stem cells obtained from adult cells, preferably derived from peripheral blood mononuclear cells.

3. The cells of any of claim 1 or 2, wherein the metabolic disease is pyruvate kinase deficiency (PKD).

4. The cells of claim 3, wherein the gene editing is performed via a knock-in strategy by using a therapeutic matrix comprising a partial codon-optimized (cDNA) RPK gene covering exons 3 to 11 preceded by a splice acceptor signal, wherein these elements are flanked by two homology arms matching sequences in the target locus of the PKLR gene, and wherein this matrix is introduced by homologous recombination in the target locus of the PKLR gene.

5. The cells of claim 4, wherein said target locus is the second intron of the PKLR gene.

6. The cells of any of claim 4 or 5, wherein the therapeutic matrix further comprises a positive-negative selection cassette preferably comprising a puromycin (Puro) resistance/thymidine (TK) fusion gene driven by a phosphoglycerate kinase promoter, wherein said positive-negative selection cassette is located downstream of the partial codon-optimized (cDNA) RPK gene.

7. A process for correcting, by gene-editing via a knock-in strategy, in cells isolated from a subject suffering from a metabolic disease affecting the erythroid lineage, the mutation or mutations in the gene causing the metabolic disease present in said cells, wherein said cells have the ability to differentiate into the erythroid lineage; and wherein said process comprises the steps of:
correcting the mutation or mutations in the gene causing the metabolic disease present in the cells by gene-editing via a knock-in strategy where a partial cDNA is inserted in a target locus of the gene causing the metabolic disease to express a chimeric mRNA formed by endogenous first exons and partial cDNA under the endogenous promoter control, wherein preferably gene-specific nucleases are used to promote homologous recombination (HR); and
optionally, collecting the knock-in cells.

8. The process according to claim 7, wherein said cells are i) hematopoietic stem or progenitor cells or ii) induced pluripotent stem cells obtained from adult cells, preferably derived from peripheral blood mononuclear cells.

9. The process according to claim 8, wherein said cells are induced pluripotent stem cells derived from peripheral blood mononuclear cells by a process comprising the following steps:
a. culturing peripheral blood mononuclear cells, isolated from a subject suffering from a metabolic disease affecting the erythroid lineage, in a cell cuture medium and expanding these cells in the presence of thrombopoietin, FLT3L, stem cell factor, granulocyte colony-stimulating factor (G-CSF) and IL-3 to promote the maintenance and proliferation of hematopoietic progenitors and myeloid-committed cells, preferably for at least 4 days; and
b. reprogramming the cells obtained from step a), by a transduction protocol by using the Sendai viral vector platform (SeV) encoding the following four reprograming factors: OCT3/4, KLF4, SOX2 and c-MYC, and maintaning these cells preferably from 3 to 6 days, preferably in the same medium; and
c. optionally, collecting the cells.

10. The process according to any of claim 7 or 8,
wherein the metabolic disease is pyruvate kinase deficiency (PKD) and the gene is the PKLR gene, and wherein the PKLR gene is gene-edited via a knock-in strategy by using a therapeutic matrix comprising a partial codon-optimized (cDNA) RPK gene covering exons 3 to 11 preceded by a splice acceptor signal, wherein these elements are flanked by two homology arms matching sequences in the target locus of the PKLR gene and wherein this matrix is introduced by homologous recombination (HR) in the target locus of the PKLR gene, wherein preferably gene-specific nucleases are used to promote HR.

11. The process according to claim 10, wherein said target locus is the second intron of the PKLR gene.

12. The process according to any of claim 10 or 11, wherein said nuclease is a PKLR transcription activator-like effector nucleases (TALEN), preferably wherein said nuclease is a PKLR TALEN which comprises two subunits defined by SEQ ID NO:1 and SEQ ID NO:2.

13. The process according to any of claims 10 to 12, wherein said nuclease is used as mRNA, preferably with 5′ and/or 3′ modifications, more preferably wherein SEQ ID NO:3 has been added in the 5′ end and/or SEQ ID NO:4 has been added in the 3′ end.

14. The process according to any of claims 10 to 13, wherein said cells are induced pluripotent stem cells derived from peripheral blood mononuclear cells by a process comprising the following steps:
a. culturing peripheral blood mononuclear cells, isolated from a subject suffering from pyruvate kinase deciency (PKD), in a cell cuture medium and expanding these cells in the presence of thrombopoietin, FLT3L, stem cell factor, granulocyte colony-stimulating factor (G-CSF) and IL-3 to promote the maintenance and proliferation of hematopoietic progenitors and myeloid-committed cells, preferably for at least 4 days; and
b. reprogramming the cells obtained from step a), by a transduction protocol by using the Sendai viral vector platform (SeV) encoding the following four reprograming factors:
OCT3/4, KLF4, SOX2 and c-MYC, and maintaning these cells preferably from 3 to 6 days, preferably in the same medium; and
c. optionally, collecting the cells.

15. Cells obtained or obtainable by the process of claims 7 to 9.

16. Cells obtained or obtainable by the process of any of claims 10 to 14.

17. The cells of any of claims 1 to 6 or 15 to 16, for its use in therapy.

18. The cells of any of claim 1-2 or 15, for its use in the treatment of a metabolic disease affecting the erythroid lineage.

19. The cells of any of claim 3 to 6 or 16, for its use in the treatment of pyruvate kinase deficiency (PKD).

20. A therapeutic matrix comprising a partial codon-optimized (cDNA) RPK gene covering exons 3 to 11 preceded by a splice acceptor signal, wherein these elements are flanked by two homology arms matching sequences in a target locus of the PKLR gene, and wherein this matrix is capable of introducing itself by homologous recombination in a target locus of the PKLR gene, preferably in the second intron of the PKLR gene.

21. The therapeutic matrix of claim 20, wherein it further comprises a positive-negative selection cassette preferably comprising a puromycin (Puro) resistance/thymidine (TK) fusión gene driven by a phosphoglycerate kinase promoter, wherein said positive-negative selection cassette is located downstream of the partial codon-optimized (cDNA) RPK.

22. Ex vivo, or in vitro, use of the therapeutic matrix of any of claim 20 or 21, for correcting, by gene-editing via a knock-in strategy, the mutation or mutations in the PKLR gene present in induced pluripotent stem cells derived from peripheral blood mononuclear cells of the erythroid lineage isolated from a subject suffering from pyruvate kinase deficiency (PKD).

23. A PKLR transcription activator-like effector nuclease (TALEN) which comprises a left subunit defined by SEQ ID NO:1 and a right subunit defined by SEQ ID NO:2.
